(19) United States
(12) Patent Application Publication
(10) Pub. No.: US 2010/0204055 A1
(43) Pub. Date: Aug. 12, 2010

(54) AUTOANTIBODY DETECTION SYSTEMS AND METHODS

(76) Inventors: Phoebe W. Bonner-Ferraby, Encinitas, CA (US); Henry Hepburne-Scott, Encinitas, CA (US); Ann McCormick, Encinitas, CA (US)

Correspondence Address: BioTechnology Law Group;12707 High Bluff Drive, Suite 200, San Diego, CA, 92130-2037, US

(21) Appl. No.: 12/631,824

(22) Filed: Dec. 5, 2009

Related U.S. Application Data
(60) Provisional application No. 61/120,335, filed on Dec. 5, 2008.

(30) Foreign Application Priority Data
Dec. 5, 2009 (US) PCT/US09/66902

(51) Int. Cl. C40B 30/04; C40B 40/10

(52) U.S. Cl. 506/9; 506/18

(57) ABSTRACT

Autoantibodies in biological samples such as serum can result from changes to biomolecules (e.g., proteins, polysaccharides, and lipids) that are associated with disease. Such autoantibodies are useful biomarkers because they frequently appear early in disease and are readily accessible, particularly in biological fluids such as blood and serum. CT antigens are particularly useful for detecting autoantibodies correlated with cancer. Numerous population-based profiles for pluralities of different autoantibody species, at least some of which are specifically reactive with CT antigens, allow for simultaneous assessment of multiple disease-associated analytes is a single test, which can be more effective in diagnostics and drug development than individual profiles. The instant invention provides autoantibody detection array devices that include a plurality of independently selected autoantibody-reactive reagent species, such as full-length CT antigens or the antigenic portions thereof, disposed on a substrate. Such arrays can be used to screen biological samples taken from patients or other subjects for diagnostic, drug development, and other applications.

### RELATED APPLICATIONS

This patent application claims priority to U.S. provisional patent application Ser. No. 61/120,335, filed 5 Dec. 2008 (attorney docket number SER-1001-PV), and PCT patent application serial number PCT/U.S.09/66902, filed 5 Dec. 2009 (attorney docket number SER-1001-PC). Each of these applications is hereby incorporated by reference in its entirety for any and all purposes.

### TECHNICAL FIELD

This invention concerns devices and methods for the detection of autoantibodies in biological samples, as well as to ways of using data and information generated through the use of such devices and methods.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

Serum autoantibodies are known, and have been proposed for use in diagnostics, prognostics, companion diagnostics, and drug discovery and development. They have also been posited as possibly being useful in connection with autoimmune diseases and cancer. Their potential usefulness in the cancer arena has been enhanced with the discovery of Cancer-Testis (CT) antigens, which are immunogenic and absent in normal, healthy adult tissue. The ability to assay human serum for antibodies to CT antigens, whether now known or later discovered, may therefore prove valuable. However, to date few applications for autoantibodies to CT antigens have been found in diagnostics and drug development due to various difficulties.

This invention overcomes existing hurdles that have prevented the exploitation of autoantibodies to CT antigens for diagnostic and drug development applications. In particular, the invention involves immobilizing a plurality of CT antigens on a substrate (preferably a solid support) that can then be used to probe biological samples to determine, for example, the autoantibody profile of the sample. Such devices, comprising an array that includes a number of different CT antigens, alone or in conjunction with moieties and/or reagents capable of detecting other biologically significant analytes, allow highly reproducible assays to be performed, including those run in multiplex or high-throughput formats.

### 3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “risk” relates to the possibility or probability of a particular event occurring either presently, or, at some point in the future. “Risk stratification” refers to an arraying of known clinical risk factors to allow physicians to classify patients into a low, moderate, high or highest risk of developing of a particular disease, disorder, or condition.

“Diagnosing” includes determining, monitoring, confirmation, subclassification, and prediction of the relevant disease, complication, or risk. “Determining” relates to becoming aware of a disease, complication, risk, or entity (e.g., autoantibody). “Monitoring” relates to keeping track of an already diagnosed disease, complication, or risk factor, e.g., to analyze the progression of the disease or the influence of a particular treatment on the progression of disease or complication. “Confirmation” relates to the strengthening or substantiating of a diagnosis already performed using other indicators or markers. “Classification” or “subclassification” relates to further defining a diagnosis according to different subclasses of the diagnosed disease, disorder, or condition, e.g., defining according to mild, moderate, or severe forms of the disease or risk. “Prediction” relates to prognosing a disease, disorder, condition, or complication before other symptoms or markers have become evident or have become significantly altered.

A “subject” is a member of any animal species, preferably a mammalian species, optionally a human. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology. The subject can be an apparently healthy individual, an individual suffering from a disease, or an individual being treated for a disease. A “reference subject” or “reference subjects” is/are an individual or a population that serves as a reference against which to assess another individual or population with respect to one or more parameters.

The term “normal” or “clinically normal” means the subject has no known or apparent or presently detectable disease or dysfunction and no detectable increase in autoantibodies to endogenous antigens correlated with a disease, particularly a cancer.

“Samples” that can be assayed using the methods of the present invention include biological fluids, such as whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant that could contain the analyte of interest. Samples can be obtained by any appropriate method known in the art.

An “analyte” refers to the substance to be detected, which may be suspected of being present in the sample (i.e., the biological sample). The analyte can be any substance for which there exists a naturally occurring specific binding partner or for which a specific binding partner can be prepared. Thus, an analyte is a substance that can bind to one or more specific binding partners in an assay.

A “binding partner” is a member of a binding pair, i.e., a pair of molecules wherein one of the molecules binds to the second molecule. Binding partners that bind specifically are termed “specific binding partners.” In addition to antigen and antibody binding partners commonly used in immunoassays, other specific binding partners can include biotin and avidin (or streptavidin), carbohydrates and lectins, nucleic acids with complementary nucleotide sequences, effector and receptor molecules, cofactors and enzymes, enzyme inhibitors and enzymes, and the like. Furthermore, specific binding partners can include partner(s) that is/are analog(s) of the original specific binding partner, for example, an analyte-analog. Immunoreactive specific binding partners include antigens, antigen fragments, antibodies and antibody fragments, both monoclonal and polyclonal, and complexes thereof, including those formed by recombinant DNA methods.

As used herein, the term “epitope” or “epitopes,” or “epitopes of interest” refer to a site(s) on any molecule that is recognized and is capable of binding to a complementary site(s) on its specific binding partner. The epitope-bearing molecule and specific binding partner are part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner, can be, but is not limited to, an antibody, e.g., an autoantibody. Typically an epitope is contained within a larger molecular framework (e.g., in the context of an antigenic region of a protein, the epitope is the region or fragment of the protein having the structure capable of being bound by an antibody reactive against that epitope) and refers to the precise residues known to contact the specific binding partner. As is known, it is possible for an antigen or antigenic fragment to contain more than one epitope.

As used herein, “specific” or “specificity” in the context of an interaction between members of a specific binding pair (e.g., an antigen and antibody) refers to the selective reactivity of the interaction. The phrase “specifically binds to” and analogous terms thereof refer to the ability of autoantibodies to specifically bind to (e.g., preferentially react with) an endogenous antigen and not specifically bind to other entities. Antibodies (including autoantibodies) or antibody fragments that specifically bind to an endogenous antigen correlated with cancer can be identified, for example, by diagnostic immunoassays (e.g., radioimmunoassays (“RIA”) and enzyme-linked immunosorbent assays (“ELISAs”), surface plasmon resonance, or other techniques known to those of skill in the art. In one embodiment, the term “specifically binds” or “specifically reactive” indicates that the binding preference (e.g., affinity) for the target analyte is at least about 2-fold, more preferably at least about 5-fold, 10-fold, 100-fold, 1,000-fold, a million-fold or more over a non-specific target molecule (e.g., a randomly generated molecule lacking the specifically recognized site(s)).

An antigen, antibody, or other analyte “correlated” or “associated” with a disease, particularly cancer refers to an antigen antibody, or other analyte as the case may be that is positively correlated with the presence or occurrence of cancer generally or a specific type of cancer, as the context requires. In general, an “antigen” is any substance that exhibits specific immunological reactivity with a target antibody, which, in the context of the present invention, is generally an autoantibody (i.e., an antibody produced naturally by the subject's own immune system). Suitable antigens, particularly CT antigens, may include, without limitation, molecules comprising at least one antigenic epitope capable of interacting specifically with the variable region or complementarity determining region (CDR) of an antibody or CDR-containing antibody fragment. Antigens typically are naturally occurring or synthetic biological macromolecules such as a protein, peptide, polysaccharide, lipids, or nucleic acids, or complexes containing these or other molecules.

A “Cancer-Testis” or “CT” antigen is an immunogenic protein preferentially expressed in normal gametogenic tissues and different histological types of tumors. The practical importance of these proteins is that due to their restricted expression pattern they are frequently recognized by the immune system of cancer patients.

An autoantibody which is described as being directed “to a different endogenous antigen correlated with cancer” means that the particular autoantibody species has specificity to a different endogenous antigen, or variant form of the endogenous antigen, and is not merely directed to a different epitope in the same endogenous antigen. However, in addition to the method and panel in the test kit being designed for assessing autoantibodies to different endogenous antigens correlated with disease (e.g. cancer), optionally, the method and the test kit panel can include means for the detection of one or more autoantibodies which are directed to the same endogenous antigen. In other words, it may be desirable to include in a panel multiple epitopes or antigenic sites from a particular endogenous antigen for detecting autoantibodies, particularly when the endogenous antigen is a complex antigenic molecule.

As used herein with reference to autoantibodies to endogenous cancer (or other disease-associated) antigens (or other analytes correlated with cancer or other disease), the term “elevated level” refers to a level in a sample that is higher than a normal level or range, or is higher that an other reference level or range (e.g., earlier or baseline sample). The term “altered level” refers to a level in a sample that is altered (increased or decreased) over a normal level or range, or over another reference level or range (e.g., earlier or baseline sample). The normal level or range for endogenous cancer antigens (e.g., CT antigens) and autoantibodies reactive therewith is defined in accordance with standard practice. Because the levels of antibodies in some instances will be very low, a so-called altered level or alteration can be considered to have occurred when there is any net change as compared to the normal level or range, or reference level or range that cannot be explained by experimental error or sample variation. Thus, the level measured in a particular sample will be compared with the level or range of levels determined in similar samples of normal tissue. In this context, “normal tissue” is tissue from an individual with no detectable cancer pathology, and a “normal” (sometimes termed “control”) patient (i.e., subject) or population is one that exhibits no detectable pathology. The level of an analyte is said to be “elevated” where the analyte is normally undetectable (e.g., the normal level is zero, or within a range of from about 25 to about 75 percentiles of normal populations), but is detected in a test sample, as well as where the analyte is present in the test sample at a higher than normal level.

An “array” refers a device consisting of a substrate, typically a solid support having a surface adapted to receive and immobilize a plurality of different protein, peptide, and/or nucleic acid species (i.e., capture or detection reagents) that can used to determine the presence and/or amount of other molecules (i.e., analytes) in biological samples such as blood. A “microarray” refers to an array wherein the different detection reagents disposed on the substrate.

The term “solid phase” refers to any material or substrate that is insoluble, or can be made insoluble by a subsequent reaction. A solid phase can be chosen for its intrinsic ability to attract and immobilize a capture or detection reagent. Alternatively, a solid phase can have affixed thereto a linking agent that has the ability to attract and immobilize a capture agent. The linking agent can, for example, include a charged substance that is oppositely charged with respect to the capture agent itself or to a charged substance conjugated to the capture agent. In general, a linking agent can be any binding partner (preferably specific) that is immobilized on (said to be “attached to”) a solid phase and that has the ability to immobilize a desired capture or detection reagent through a binding or other associative reaction. A linking agent enables the indirect binding of a capture agent to a solid phase material before the performance of an assay or during the performance of an assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

As used herein, term “microparticle” refers to a small particle that is recoverable by any suitable process, e.g., magnetic separation or association, ultracentrifugation, etc. Microparticles typically have an average diameter on the order of about 1 micron or less.

A “capture” or “detection” agent or reagent refers to a binding partner that binds to an analyte, preferably specifically. Capture or detection reagents can be attached to or otherwise associated with a solid phase.

The term “labeled detection agent” refers to a binding partner that binds to an analyte, preferably specifically, and is labeled with a detectable label or becomes labeled with a detectable label during use in an assay. A “detectable label” includes a moiety that is detectable or that can be rendered detectable. With reference to a labeled detection agent, a “direct label” is a detectable label that is attached, by any means, to the detection agent, and an “indirect label” is a detectable label that specifically binds the detection agent. Thus, an indirect label includes a moiety that is the specific binding partner of a moiety of the detection agent. Biotin and avidin are examples of such moieties that can be employed, for example, by contacting a biotinylated antibody with labeled avidin to produce an indirectly labeled antibody.

The term “indicator reagent” refers to any agent that is contacted with a label to produce a detectable signal. Thus, for example, in conventional enzyme labeling, an antibody labeled with an enzyme can be contacted with a substrate (the indicator reagent) to produce a detectable signal, such as a colored reaction product.

An “antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Antibodies are generally found in bodily fluids, mainly blood.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the (Fab′)₂ dimer into a Fab′ monomer. The Fab′ monomer is essentially a Fab with part of the hinge region. While various antibody fragments are defined in terms of the digestion of an intact antibody, one of skill will appreciate that such Fab′ fragments may be synthesized de novo either chemically or by utilizing recombinant DNA methodology. Thus, in the context of the invention the term “antibody” also includes antibody fragments either produced by the modification of whole antibodies or synthesized de novo using recombinant DNA methodologies. Antibodies include single chain antibodies (antibodies that exist as a single polypeptide chain), single chain Fv antibodies (sFv or scFv), in which a variable heavy and a variable light chain are joined together (directly or through a peptide linker) to form a continuous polypeptide. The single chain Fv antibody is a covalently linked VH-VL heterodimer that may be expressed from a nucleic acid including VH- and VL-encoding sequences either joined directly or joined by a peptide-encoding linker. While the VH and VL are connected to each as a single polypeptide chain, the VH and VL domains associate non-covalently. The scFv antibodies and a number of other structures convert the naturally aggregated, but chemically separated, light and heavy polypeptide chains from an antibody V region into a molecule that folds into a three dimensional structure substantially similar to the structure of an antigen-binding site are known to those of skill in the art.

An “autoantibody” is a naturally occurring antibody that binds to an analyte that occurs in the individual in which the antibody is produced because the individual's immune system recognizes the analyte (typically a protein or polypeptide) as foreign even though that antigen actually originated in the individual. Generally the analyte is one that occurs naturally in a subject, including analytes that are the result of or arise from a disease or disease process (e.g., altered forms of naturally occurring proteins produced by a diseased cell or during a disease process) as well as those that result from vaccination. An autoantibody to an endogenous cancer-correlated antigen is an autoantibody produced by the subject's immune system that binds an endogenous antigen correlated with occurrence of disease, e.g., cancer.

An “autoantibody-reactive reagent species” is a molecule, or complex of two or more molecules, specifically reactive with an autoantibody correlated with a disease-associated target antigen species, such as a tumor antigen, for example, a CT antigen.

A “panel” refers to a group of two or more distinct molecular species that have shown to be indicative of or otherwise correlated with a particular disease or health condition. Such “molecular species” may be referred to as “biomarkers”, with the term “biomarker” being understood to mean a biological molecule the presence or absence of which serves as an indicator of a particular biological state, for example, the occurrence (or likelihood of the occurrence) of cancer in a subject. In other words, a biomarker is a characteristic that can objectively measured and evaluated as an indicator of normal biologic processes, pathogenic processes, or pharmacologic responses to a therapeutic intervention (see 1998 definition from NIH study group). In the context of the invention an “assay panel” or “array panel” refers to an article, typically a solid phase substrate, having a panel of capture reagents associated therewith (typically by immobilization), wherein at least on of the capture reagents is specifically reactive with an endogenous cancer antigen (e.g., a CT antigen). In some embodiments, an assay panel includes 1, 2, 3, 4, 5, 6, 7, 8, 9, 10, 11, 12, 13, 14, 15, 16, 17, 18, 19, 20, or more (e.g., 25, 30, 35, 40, 50, 75, 100, 150, 200, 250, 500, etc., including any integer, or range of integers from 1 to 500) different detection reagents that are proteinaceous cancer-associated antigens (e.g., CT antigens), alone or combination with other detection reagents (e.g., nucleic acid-based detection reagents, etc.) correlated with the presence of disease (e.g., cancer) in a subject.

A “biological sample” is a sample of biological material taken from a patient or subject. Biological samples include samples taken from bodily fluids and tissues (e.g., from a biopsy) or tissue preparations (e.g., tissue sections, homogenates, etc.). A “bodily fluid” is any fluid obtained or derived from a subject suitable for use in accordance with the invention. Such fluids include whole blood, blood fractions such as serum and plasma, urine, sweat, lymph, feces, ascites, seminal fluid, sputum, nipple aspirate, post-operative seroma, wound drainage fluid, saliva, synovial fluid, bone marrow, cerebrospinal fluid, nasal secretions, amniotic fluid, bronchoalveolar lavage fluid, peripheral blood mononuclear cells, total white blood cells, lymph node cells, spleen cells, and tonsil cells.

A “companion diagnostic” is a diagnostic test designed to identify subgroups of patients who may or may not benefit from a particular drug, who may have adverse reactions to the drug, or may require different dosages of the drug.

The term “drug rescue” refers to a drug or drug candidate in the context of the reevaluation of samples and/or data from discontinued clinical trials or pre-clinical development with new or improved evaluation methods.

The term “high-throughput” refers to the ability to rapidly process multiple specimens, for example, arrays or microarrays according to the invention, in an automated and/or massively parallel manner. On the other hand, the term “multiplex” refers to the concurrent performance of multiple experiments on a single device or in a single assay. For instance, a multiplex assay using an array according to the invention allows the simultaneous detection and/or measurement of a plurality of different autoantibody species in a biological sample on a single device.

A “patentable” process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

The term “sample profiling” refers to a representation of information relating to the characteristics of a biological sample, for example, serum, recorded in a quantified way in order to determine patterns or signatures of biomolecules (e.g., autoantibodies correlated with the presence or absence of cancer or other disease) in the particular sample.

As used herein, the singular forms “a”, “an”, and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

### SUMMARY OF THE INVENTION

It is an object of the invention to provide articles, kits, and methods for the multiplex detection of disease-associated autoantibodies in biological samples obtained from subjects, including sera sampled from patients. As described herein, simultaneous assessment of two or more naturally occurring autoantibody species to cancer-associated antigens, for example, at least one of which is a Cancer/Testis (CT) antigen, can be used for drug development and for diagnosis, prognosis, risk stratification, staging, monitoring, categorizing and determination of further diagnosis and treatment regimens in subjects suffering or at risk of suffering from cancer, metastasis, or disease recurrence.

Thus, one aspect of the invention concerns autoantibody detection panels that include detection reagents for at least two cancer-associated autoantibody species, of which at least one autoantibody species is specifically reactive with a naturally occurring CT antigen. If desired, detection reagents for other analyte classes (e.g., nucleic acids, lipids, carbohydrates and polysaccharides, non-antibody proteins, etc.) can also be included in panels of the invention.

In general, an autoantibody detection reagent of the invention includes a moiety, typically a peptide or polypeptide specifically reactive with a target disease-associated autoantibody species present, for example, in a serum sample obtained from a patient. The autoantibody-specific moiety is preferably immobilized on a substrate, which can be any solid support suitable for immunoassay-based analyses. The choice of a particular substrate depends on many factors, such as the assay format, the number of analytes to be assayed for, the moieties used as autoantibody detection reagents, etc.

In certain preferred embodiments, the autoantibody detection panels of the invention employ autoantibody detection reagents that comprise recombinantly expressed full-length versions of the naturally occurring antigens with which the target autoantibody species react in vivo. With regard to autoantibody detection reagents for anti-CT antigen serum antibodies, i.e., CT antigen reagent species, in particularly preferred embodiments such detection reagents are expressed via recombinant techniques in a suitable eukaryotic expression system, for example, mammalian expression systems such as those based on recombinant CHO (Chinese Hamster Ovary) cells or the human cell line PER.C6, as well as insect cell-based expression systems, so that authentic CT antigen epitopes are exhibited. Of course, such systems, as well as solid-state synthetic processes, can also be used to produce partial proteins and protein fragments (including truncated proteins where one or more N- and/or C-terminal amino acid residues or domains are absent) and peptides of the autoantibody-specific antigens can also be used for autoantibody detection. Similarly, panels that employ one or more engineered or otherwise optimized autoantibody detection reagent species are also. For example, an autoantibody detection reagent that includes as an autoantibody-reactive moiety a polypeptide in which the antigenic epitope has been affinity matured or otherwise modified (e.g., by phage display techniques), can also be used. In certain embodiments, systems that employ automated liquid handling approaches are adapted for use in practicing the invention in order to allow for high throughput, multiplex well-controlled assay performance.

In the context of the invention, a panel comprising at least one CT antigen (or a derivative thereof) and at least one cancer-associated non-CT antigen (or a derivative thereof) can be used for cancer diagnosis (i.e., to screen for an initial occurrence, recurrence, progression, or metastasis), for risk stratification (that is, to identify subjects at risk for developing cancer or undergoing progression, metastasis, relapse, or recurrence of an already-diagnosed cancer); for monitoring for deterioration or improvement of clinical status; and for predicting a future medical outcome, such as improved or worsening disease, a decreased or increased mortality risk, or responsiveness to a particular therapeutic regimen.

In another aspect, the present invention relates to methods for evaluating a biological sample from a subject to assess whether it contains two or more autoantibody species associated with cancer, wherein at least one of the autoantibody species is specifically reactive with a CT antigen. These methods comprise performing an assay configured to detect autoantibody species in a biological sample, such as a body fluid, obtained from a subject. The assay result, for example, a measured level of serum antibodies to two different CT antigens, is then correlated with the presence or absence of cancer, and may be used for one or more of risk stratification, diagnosis, prognosis, staging, classifying, monitoring, and treatment. Thus, the present invention utilizes panels that comprise reagents to detect at least two or more autoantibody species correlated with cancer.

In various related aspects, the present invention also relates to devices and kits for performing the methods described herein. Suitable kits comprise reagents sufficient for performing an assay according to the invention, together with instructions for performing the described threshold comparisons

Features and advantages of the invention will be apparent from the following drawings, detailed description, and appended claims.

### BRIEF DESCRIPTION OF THE FIGURES

FIG. 1: Patients A and B have both been diagnosed with colorectal cancer. One patient is at the more advanced stage IV versus the other patient's stage II disease.

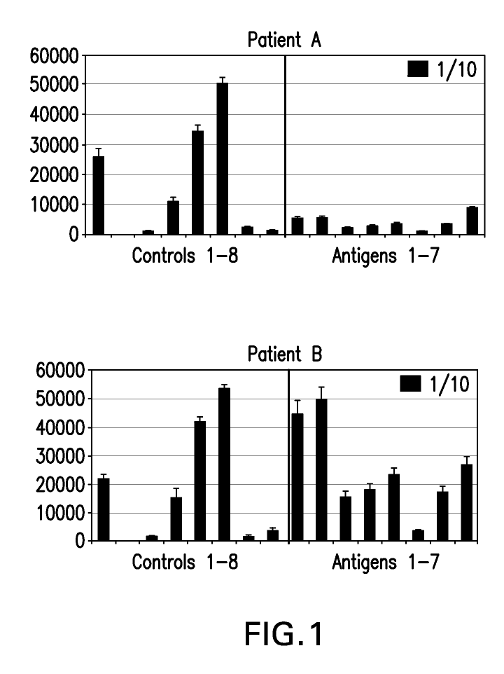

FIG. 2: An overview of an assay according to the invention.

FIG. 3: Sensitivity and dynamic range of antibody detection using a protein microarray according to the invention.

FIG. 4: A plot of results from Example 2.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

More specifically, the present invention relates to articles, devices, kits, and methods for diagnosis, differential diagnosis, risk stratification, monitoring, classifying, and determination of treatment regimens in subjects suffering or at risk of suffering from cancer through measurement of a plurality of biomarkers, particularly serum autoantibodies correlated with cancer. At least one of the autoantibody species to be measured is specifically reactive to a CT antigen.

### Biomarker Changes in Disease

The cellular changes that mark the transition from a healthy to a diseased state are frequently, if not always, mediated by changes in the level or type of constituent biomarkers, including proteins, nucleic acids, carbohydrates, and lipids. These changes can result from several different mechanisms, including changes in the abundance or expression level of certain proteins, the rate of transcription of DNA to mRNA or the translation of mRNA to protein, mRNA stability, the rate of protein turnover, or other metabolic processes. One, some, or all of these and other mechanisms may be modulated, with the result being that the synthesis and/or stability of one or more biomarker species is increased or decreased in a manner that can be detected in an assay of a biological sample. With particular regard to proteins, there may also be changes in the primary sequence of a protein conferred by alterations in the corresponding gene sequences, due to single nucleotide polymorphisms (SNPs), alternate mRNA splicing, genomic rearrangements, or any of several other mechanisms for genetic variation. There may also be changes in the processing and post-translational modification of proteins. For example, a protein may be differentially glycosylated such that alternative glycoforms can be detected.

### Autoantibodies in Disease

Autoantibody detection is well suited for non-invasive or minimally invasive diagnostic applications, and since autoantibodies are generally abundant relative to diseased tissue (amplification effect), their detection can give earlier warning of disease than other biomarker classes. For instance, tumors typically express certain proteins that enable them to become malignant. Often, these newly expressed proteins provoke an autoantibody response. An autoantibody response can occur very early during tumorigenesis, often before a tumor can otherwise be detected. Accordingly, detection of autoantibodies to tumor antigens is attractive for various diagnostic applications, including patient screening and monitoring, prognosis, monitoring of disease progression and/or response to treatment, etc., as well as for drug development (for example, as a “companion diagnostic” that identifies patient sub-populations that detrimentally or favorably respond, and/or fail to respond, to a particular drug, drug candidate, or combinations thereof).

The functions of the adaptive immune system, including the recognition of non-self proteins and the humoral immune response, can result in the production of serum antibodies to “foreign” antigens. Proteins may be recognized as “non-self” for a variety of reasons even when they are self-derived and not the result of an infection by a foreign microorganism (including bacteria, fungi, protozoa, and viruses). Abnormal glycoforms, non-native folding, or protein variants that result from alternative splicing of mRNA species, as well as variants that result from mutation, can result in the presentation of novel epitopes that can be immunogenic, or at least antigenic. For example, in autoimmune disease the mounting of an immune response to self-antigens is harmful because it results in destruction of otherwise healthy tissue, such as myelin in multiple sclerosis, or histones in lupus. In cancer, however, the recognition of self-antigens as “non-self” by the immune system can be beneficial because it may lead to or result, for example, in destruction of tumors or cancerous cells. And when the humoral immune response does not result in tumor destruction, as can happen, for example, when the tumor represses the cellular arm of the immune response, serum autoantibodies may still be present. Their presence can thus be exploited, for instance, in diagnostic and drug development applications.

For a particular disease, serum antibody (i.e., “autoantibody”) profiles from normal samples (i.e., suitable negative control samples obtained from patients known not to have the particular disease) and diseased samples (i.e., samples obtained from patients known to have the particular disease, whether by clinical manifestation, histology, or immunohistochemistry, etc.) can be compared and used to define a panel of disease-specific autoantibodies the detection of which can be used for diagnostic, prognostic, and/or treatment purposes. Matched normal serum controls provide insight into patients' humoral immune responses to disease or disease progression.

### CT Antigens in Cancer

Early placenta (trophoblast) needs to perform normal trophoblastic functions such as invading the endometrial wall, securing a blood supply for itself (angiogenesis), overcoming mother's immune system, etc. Proteins mediate many of these functions. The functions of many of the proteins expressed in primitive embryonic/trophoblastic can also be also useful to tumors, which is why in many tumor cell types these long-quiescent genes are expressed.

Because some of the proteins expressed in tumors are otherwise only expressed, if at all, in one or more stages of early development, the immune system of an infant, child, adolescent, or adult may not be tolerized to them. Thus, should they subsequently be expressed, for example, during tumorigenesis or metastasis as a result of dysregulation of gene expression (for example, due to mutation, chromosomal rearrangement, and the like), an antibody response reactive against these proteins may ensue in the subject.

It has been shown that autoantibodies to tumor antigens can be found in biological samples obtained from cancer patients, including in patient serum. Many of these autoantibody species are known, as are the antigens that can give rise to the production of autoantibodies specifically reactive to such antigens. A number of autoantibody species and their corresponding antigens have been reported in the SEREX (serological expression of cDNA expression libraries) database (Ludwig Institute for Cancer Research Ltd; www.licr.org). The SEREX database comprises a list of antigens that have been shown to elicit an antibody response in cancer by probing tumor-derived cDNA libraries with autologous serum. The inventors have appreciated that autoantibodies to antigens referenced in the SEREX database or which are now or subsequently become known are important predictive cancer diagnostics because they are produced early during disease, long before the tumor may be detectable clinically or through imaging or other conventional techniques.

All autoantibodies to tumor antigens have application in the context of the invention, including for diagnostic and drug development applications. A sub-set of these antigens, however, the CT (Cancer-Testis) antigens, are of particular importance in the context of this invention. CT antigens comprise a subset of the antigens reported in the SEREX database. The CT antigens have particular value because they are generally absent, or detectable only at extremely low levels, in normal tissue, as their expression is generally believed to be restricted to germ cells (i.e., cells of the testis, ovary, and trophoblast); they are not believed to be expressed in appreciable, physiologically relevant levels in normal adult somatic tissues. An example of a CT antigen exploited for drug development is MAGE-A3, a protein expressed in 35-50% of patients diagnosed with early-stage non-small cell lung carcinoma (NSCLC), as well as in patients diagnosed with head and neck cancer and bladder cancer. A highly purified recombinant form of MAGE-A3 has been developed, which when combined appropriate adjuvant formulations, may be useful in stimulating a patient's own immune system to react to MAGE-A3-positive tumor cells, including NSCLC tumor cells, to produce a specific anti-tumor immune response. Such compounds may be useful in reducing risk of tumor recurrence following surgical tumor resection or to minimize tumor growth in early phases of metastasis.

Although to date a few CT antigens have been targeted individually in drug development efforts, they have generally not been employed for diagnostic applications, particularly in multiplex formats. The inventors have appreciated that the ability to detect autoantibodies to multiple CT antigens simultaneously in a single assay would be advantageous as it would allow population-based autoantibody profiles to be developed for normal as compared to diseased organisms, as opposed to having to assess changes over time to individualized autoantibody profiles, although assessing changes to an individual's autoantibody profile over time is also within the scope of the invention (and can be used, for example, to monitor disease progression, to assess treatment efficacy, for prognostic applications, etc.). Population-based autoantibody profiles are much more effective in diagnostic and drug development applications. In addition, parallel assessment of a plurality of autoantibody species (or a plurality of autoantibody species and at least one other biomarker from one or more different biomarker classes) in a single test is much more likely to be discriminatory for disease than assessment of an individual biomarker species. Furthermore, therapeutic approaches that involve vaccination against a panel of antigens rather than a single disease-associated antigen is much more likely to be effective since tumor cells, for example, are better able to evade a single-antigen vaccine through redundancy and immune suppression than a vaccine that involves a panel of disease-associated antigens.

### Autoantibody Detection

The presence and/or amount of autoantibodies can be detected or measured in biological samples obtained from subjects by any suitable method, including via biopsy, a buccal swab, or other technique useful to collect a biological fluid or tissue from a patient. Particularly preferred biological samples are patient sera, which has several advantages when compared to other, more conventional biomarker classes. Firstly, serum is a readily accessible tissue that can be obtained by relatively non-invasive sampling techniques. In the context of cancer, use of a fluid such as serum reduced the need to locate and access tumors for biopsy, for example. Secondly, antibodies provide an amplified response and their relative abundance enables early warning or detection of small changes detectable at the molecular level. Thirdly, serum proteins are a common and stable protein type and multiple immunoglobulin protein species can be assayed in one biomarker panel.

Autoantibodies are generally detected using autoantibody-reactive reagent species immobilized on a substrate such as a solid support. An autoantibody-reactive reagent species is specifically reactive with an autoantibody correlated with a disease-associated target antigen species, such as a tumor antigen, for example, a CT antigen. Thus, a CT antigen reagent species refers to a reagent that is specifically reactive with an autoantibody reactive against a particular CT antigen, the antigenic portion of which is represented in the particular CT antigen reagent species. In other words, an autoantibody-reactive reagent species preferably comprises the antigenic feature(s) of the naturally occurring antigen with which a naturally occurring autoantibody to the antigen will react. Preferred autoantibody-reactive reagent species are naturally occurring or recombinantly expressed full-length forms of known polypeptide antigens or variants or truncated forms of, or peptides derived from, naturally occurring antigens that contain one or more epitopes reactive with the corresponding autoantibody to be detected.

In this invention, at least two autoantibody-reactive reagent species, at least one of which is a CT antigen reagent species, are immobilized on a suitable substrate, for example, plastic beads, on the surface of the detection zone of a lateral flow device, etc. In this way, the autoantibody-reactive reagent species can be brought into contact with a small biological sample (e.g., from about 1 nanoliter (nL) to about 500 microliters (uL) of serum) to determine if it contains autoantibodies correlated with a disease or disorder, for example, cancer.

An autoantibody detection array (or other configuration of multiple autoantibody-reactive reagent species immobilized on one or more substrates) of the invention can also include other moieties reactive with biomolecules in a biological sample. For example, detection reagents reactive with disease-associated metabolites, proteins, and/or nucleic acids that encode them, can also be included. Representative examples of other suitable non-CT antigen biomarkers include metabolites such as sacrosine (an N-methyl derivative of the amino acid glycine, the level of which markedly rises in men having prostate cancer), mRNA for PCA3 (prostate cancer antigen 3), nucleic acids encoding TMPRSS2-ERG (which results from the fusion of the TMPRSS2 and ERG genes in at least 50% of human prostate cancers), and p53, matrix metalloproteinases, and mucins, etc. Detection reagents for these and/or other disease-associated biomarkers can also be included in a panel or on an array according to the invention.

In preferred embodiments, the arrays of the invention comprise at least two autoantibody-reactive reagent species, each of which corresponds to a specific CT antigen.

Tables 1 and 2, below, set out lists of preferred autoantibody antigens. Non-CT antigens associated or correlated with disease, particularly one or more cancers, are listed in Table 1 and CT antigens are listed in Table 2. Additional CT and non-CT antigens not listed below or discovered in the future may also be used in the practice of this invention.

TABLE 1
Non-CT Antigens associated with cancer

| | | | Disease |
|---|---|---|---|
| Gene Family | Family Member | Ref Seq | Association/Comment |
| AKT1 | AKT1 | NM_005163 | Kinase |
| AKT1 | AKT1 | NM_001014431 | Kinase |
| CALM1 | CALM1 | NM_006888 | Cancer Protein |
| Caspase8 | Caspase-8 | NM_033355 | |
| CDC25A | CDC25 | NM_201567 | Cancer Protein |
| CDK2 | CDK2 | NM_001798 | Kinase |
| CDK4 | CDK4 | NM_000075 | Kinase |
| CDK7 | CDK7 | NM_001799 | Kinase |
| CDKN2B | CDKN2B | NM_078487 | |
| COL6A1 | COL6A1 | NM_001848 | Breast cancer |
| CREB1 | CREB1 | NM_134442 | Cancer Protein |
| CREB1 | CREB1 | NM_004379 | Cancer Protein |
| CTNNB1 | CTNNB1 | NM_001904 | Cancer Protein |
| Cytochrome p450 3A4 | Cytochrome p450 3A4 | NM_017460 | Drug metabolizing enzyme |
| Cytochrome p450 | Cytochrome p450 | NM_000941 | Drug metabolizing accessory |
| reductase (POR) | reductase (POR) | | protein |
| EGFR (ERBB1) | EGFR (ERBB1) | NM_005228 | Kinase |
| ERBB2 | ERBB2 | NM_004448 | |
| FES | FES | NM_002005 | Kinase |
| FGFR2 | FGFR2 | NM_000141 | Kinase |
| FGFR2 | FGFR2 | NM_022970 | Kinase |
| GRWD1 | GRWD1 | NM_031485 | Breast cancer |
| LAGE3 | LAGE3 | NM_006014 | |
| LOC441294(CTAG | LOC441294(CTAG | NM_001008747 | |
| relative) | relative) | | |
| MAGED | MAGED1 | NM_001005332 | |
| MAGED | MAGED1 | NM_001005333 | |
| MAPK! | MAPK! | NM_002745 | Kinase |
| MAPK1 | MAPK1 | NM_138957 | Kinase |
| MAPK3 | MAPK3 | NM_002746 | Kinase |
| MART-1 | MART-1 | NM_005511 | Melanoma |
| MICA | MICA | NM_000247 | Melanoma |
| MICAL1 | MICAL1 | NM_022765 | |
| MICAL2 | MICAL2 | NM_014632 | |
| MICALL2 | MICALL2 | NM_182924 | |
| MMP | MMP2 | NM_031414 | |
| MUM | MUM-1 | NM_032853 | |
| p53 | p53 | NM_000546 | Tumor Suppressor |
| p53 S6A | p53 | | Cancer-associated p53 variant |
| p53 C141Y | p53 | | Cancer-associated and |
| | | | germline p53 variant |
| p53 S15A | p53 | | Cancer-associated p53 variant |
| p53 T18A | p53 | | Cancer-associated p53 variant |
| p53 Q136X | p53 | | Germline p53 variant |
| p53 S46A | p53 | | Cancer-associated p53 variant |
| p53 S46P | p53 | | Cancer-associated p53 variant |
| p53 S46F | p53 | | Cancer-associated p53 variant |
| p53 K382R | p53 | | |
| | | | Cancer-associated and |
| p53 S392A | p53 | | germline p53 variant |
| | | | Cancer-associated and |
| p53 M133T | p53 | | germline p53 variant |
| | | | Cancer-associated and |
| p53 L344P | p53 | | germline p53 variant |
| PALB2 | PALB2 | NM_024675 | |
| PRAME | PRAME | NM_006115 | |
| PRAME | PRAME | NM_206954 | |
| PRAME | PRAME | NM_206955 | |
| PRAME | PRAMEF2 | NM_023014 | |
| PRAME | PRAMEF10 | NM_001039361 | |
| PRKCZ | PRKCZ | NM_002744 | Kinase |
| RAF | RAF | NM_014943 | Kinase |
| RAGE | RAGE(AGER) | NM_001136 | |
| RELT | RELT | NM_152222 | Melanoma |
| SART | SART-1 | NM_005146 | |
| SART | SART3 | NM_014706 | |
| SART | DSE(SART2) | NM_013352 | |
| SILV | SILV | NM_006928 | Melanoma |
| SRC | SRC | NM_005417 | Kinase |
| STEAP | STEAP1 | NM_012449 | |
| STEAP | STEAP2 | NM_152999 | |
| STEAP | STEAP2 | NM_001040665 | |
| STEAP | STEAP2 | NM_001040666 | |
| STEAP | STEAP3 | NM_182915 | |
| STEAP | STEAP3 | NM_018234 | |
| STEAP | STEAP3 | NM_001008410 | |
| STEAP | STEAP4 | NM-024636 | |
| STK31 | STK31 (TDRD8 and | NM_032944 | |
| | FLJ16102 | | |
| TRP | TRP-1(PRSS1) | NM_002769 | |
| TRP | TRP-2 | NM_006267 | |
| TYR | TYR | NM_000372 | Melanoma |

TABLE 2
CT Antigens

| Gene Family | Family Member | Ref Seq | CT Identifier |
|---|---|---|---|
| ACRBP | ACRBP | NM_032489 | CT 23 |
| ACTL8 | ACTL8 | NM_030812 | CT 57 |
| ADAM | ADAM2 | NM_001464 | CT 15 |
| ADAM20 | ADAM20 | NM_003814 | CT 73.0 |
| ADAM29 | ADAM29 | NM_014269 | CT 73 |
| AKAP3 | AKAP3 | NM_006422 | CT 82 |
| AKAP4 | AKAP4 | NM_003886 | CT 99 |
| ARMC3 | ARMC3 | NM_173081 | CT 81 |
| BAGE | BAGE | NM_001187 | CT 2.1 |
| BAGE | BAGE2 | NM_182482 | CT 2.2 |
| BAGE | BAGE3 | NM_182481 | CT 2.3 |
| BAGE | BAGE4 | NM_181704 | CT 2.4 |
| BAGE | BAGE5 | NM_182484 | CT 2.5 |
| BAGE | BAGE | NM_001187 | CT 2.1 |
| BRDT | BRDT | NM_001726 | CT 9 |
| C21orf99 | C21orf99 | NM_153773 | CT 85 |
| CABYR | CABYR | NM_012189 | CT 88 |
| CAGE | DDX53 | NM_182699 | CT 26 |
| CAGE | DDX53 | NM_182699 | CT 26 |
| CAGE1 | CAGE1 | NM_205864 | CT 95 |
| CALR3 | CALR3 | NM_145046 | CT 93 |
| CCDC110 | CCDC110 | NM_152775 | CT 52 |
| CCDC33 | CCDC33 | NM_182791 | CT 61 |
| CCDC33 | CCDC33 | NM_025055 | CT 61 |
| CCDC36 | CCDC36 | NM_178173 | CT 74 |
| CEP290 | CEP290 | NM_025114 | CT 87 |
| COX6B2 | COX6B2 | NM_144613 | CT 59 |
| CPXCR1 | CPXCR1 | NM_033048 | CT 77 |
| CRISP2 | TPX1 | NM_003296 | CT 36 |
| CSAG1 | CSAG1 | NM_153478 | CT 24.1 |
| CSAG2 | TRAG3 | NM_004909 | CT 24.2 |
| CSAG2 | CSAG2 | NM_001080848 | CT 24.2 |
| CT29 | AF15q14 | NM_144508 | CT 29.1 |
| CT29 | AF15q14 | NM_170589 | CT 29.2 |
| CT45 | CT45A1 | NM_001017417 | CT 45.1 |
| CT45 | CT45A2 | NM_152582 | CT 45.2 |
| CT45 | CT45A5 | NM_001007551 | CT 45.5 |
| CT45 | CT45A4 | NM_001017436 | CT 45.4 |
| CT45 | CT45A3 | NM_001017435 | CT 45.3 |
| CT45 | CT45A6 | NM_001017438 | CT 45.6 |
| CT47 | CT47.11 | NM_173571 | CT 47.11 |
| CT47 | CT47A1 | NM_001080146 | CT 47.1 |
| CT47 | CT47B1 | NM_001145718 | CT 47.13 |
| CT47 | CT47A2 | NM_001080145 | CT 47.2 |
| CT47 | CT47A3 | NM_001080144 | CT 47.3 |
| CT47 | CT47A4 | NM_001080143 | CT 47.4 |
| CT47 | CT47A5 | NM_001080142 | CT 47.5 |
| CT47 | CT47A6 | NM_001080141 | CT 47.6 |
| CT47 | CT47A7 | NM_001080140 | CT 47.7 |
| CT47 | CT47A8 | NM_001080139 | CT 47.8 |
| CT47 | CT47A9 | NM_001080138 | CT 47.9 |
| CT47 | CT47A10 | NM_001080137 | CT 47.10 |
| CT62 | CY62 | NM_001102658 | CT 62 |
| CT62 | CT62 | NM_001102658 | CT 62 |
| CTAG2 | LAGE-1a (CTAG2) | NM_172377 | CT 6.2a |
| (variant 1) | | | |
| CTAG2 | CTAG2 (LAGE-1b) | NM_020994 | CT 6.2a |
| (variant 2) | | | |
| CTAGE1 | CTAGE1 | NM_172241 | CT 21.1 |
| CTCFL | BORIS | NM_080618 | CT 27 |
| CXorf48 | CXorf48 | NM_017863 | CT 55 |
| CXorf61 | CXorf61 | NM_001017978 | CT 83 |
| DPPA | DPPA2 | NM_138815 | CT 100 |
| DSCR8 | DSCR8 | NM_032589 | CT 25.1a |
| DSCR8 | DSCR8 | | CT 25.1A |
| FATE1 | FATE1 | NM_033085 | CT 43 |
| FMR1NB | NY-SAR-35 | NM_152578 | CT 37 |
| FTHL17 | FTHL17 | NM_031894 | CT 38 |
| GAGE | GAGE1 | NM_001468 | CT 4.1 |
| GAGE | GAGE2C | NM_001472 | CT 4.2 |
| GAGE | GAGE4 | NM_001474 | CT 4.4 |
| GAGE | GAGE5 | NM_001475 | CT 4.5 |
| GAGE | GAGE6 | NM_001476 | CT 4.6 |
| GAGE | GAGE7 | NM_021123 | CT 4.7 |
| GAGE | GAGE1 (variant 2) | NM_001040663 | CT 4.1 |
| GAGE | GAGE2A | NM_001127212 | CT 4.2 |
| GAGE | GAGE3 | N/A | CT 4.3 |
| GAGE | GAGE8 | NM_012196 | CT 4.8 |
| GOLGA | GOLGA6L2 | N/A | CT 105 |
| HAGE | DDX43 | NM_018665 | CT 13 |
| HOM-TES-85 | HOM-TES-85 | NM_016383 | CT 28 |
| HORMAD1 | HORMAD1 | NM_032132 | CT 46 |
| HORMAD2 | HORMAD2 | NM_152510 | CT 46 |
| HSPB9 | HSPB9 | NM_033194 | CT 51 |
| IL13RA2 | IL13RA2 | NM_000640 | CT 19 |
| IMP-3 | IMP-3 | NM_006547 | CT 98 |
| KDM5B | KDM5B | NM_006618 | CT 31 |
| KIAA0100 | KIAA0100/MLAA-22 | NM_014680 | CT 101 |
| KLKBL4 | KLKBL4 | NM_001080492 | CT 67 |
| LDHC | LDHC | NM_002301 | CT 32 |
| LDHC Var 2 | | NM_017448 | CT 32 |
| LEMD1 | LEMD1 | NM_001001552 | CT 50 |
| LEMD3 | LEMD3 | NM_014319 | CT 50 |
| LIP1 | LIPI | NM_198996 | CT 17 |
| LY6K | LY6K | NM_017527 | CT 97 |
| MAGEA | MAGEA1 | NM_004988 | CT 1.1 |
| MAGEA | MAGEA10 | NM_001011543 | CT 1.10 |
| MAGEA | MAGEA11 | NM_001011544 | CT 1.11 |
| MAGEA | MAGEA2 | NM_005361 | CT 1.2 |
| MAGEA | MAGEA3 | NM_005362 | CT 1.3 |
| MAGEA | MAGEA4 ver2 | NM_001011548 | CT 1.4 |
| MAGEA | MAGEA4 | NM_002362 | CT 1.4 |
| MAGEA | MAGEA4 ver4 | NM_001011549 | CT 1.4d |
| MAGEA | MAGEA5 | NM_021049 | CT 1.5 |
| MAGEA | MAGEA10 | NM_021048 | CT 1.10 |
| MAGEA | MAGEA11 | NM_005366 | CT 1.11 |
| MAGEA | MAGEA12 | NM_005367 | CT 1.12 |
| MAGEA | MAGEA2B | NM153488 | CT 1.2 |
| MAGEA | MAGEA2 Var 2 | | CT 1.2b |
| MAGEA | MAGA2 Var 3 | | CT 1.2c |
| MAGEA | MAGEA4 | NM_001011550 | CT 1.4 |
| MAGEA | MAGEA6 | NM_005363 | CT 1.6 |
| MAGEA | MAGEA6 | NM_175868 | CT 1.6 |
| MAGEA | MAGEA8 | NM_005364 | CT 1.8 |
| MAGEA | MAGEA9 | NM_005365 | CT 1.9 |
| MAGEB | MAGEB1 | NM_002363 | CT 3.1 |
| MAGEB | MAGEB5 | XM_293407 | CT 3.3 |
| MAGEB | MAGEB6 | NM_173523 | CT 3.4 |
| MAGEB | MAGEB10 | NM_182506 | CT 3 |
| MAGEB | MAGEB18 | NM_173699 | CT 3 |
| MAGEB | MAGEB1 | NM_177404 | CT 3.1 |
| MAGEB | MAGEB2 | NM_002364 | CT 3.2 |
| MAGEB | MAGEB3 | NM_002365 | CT 3.5 |
| MAGEB | MAGEB4 | NM_002367 | CT 3.6 |
| MAGEC | MAGEC2 | NM_016249 | CT 10 |
| MAGEC | MAGEC1 | NM_005462 | CT 7.1 |
| MAGEC | MAGEC3 | NM_138702 | CT 7.2 |
| MMA1 | MMA1b | | CT 25.1B |
| MORC | MORC v1 | NM_014429 | CT 33 |
| MORC | MORC v2 | NM_014941 | CT 33 |
| MORC | MORC v3 | NM_015358 | CT 33 |
| MPHOSPH1 | MPHOSPH1 | NM_016195 | CT 90 |
| NLRP4 | NLRP4 | NM_134444 | CT 58 |
| NUF2 | NUF2 | NM_145697 | CT 106 |
| NXF2 | NXF2 | NM_017809 | CT 39 |
| NXF2 | NXF2 | NM022053 | CT 39 |
| NY-ESO-1 | NY-ESO-1 | NM_001327 | CT 6.1 |
| NY-ESO-1 | LAGE2A | NM_139250 | CT 6.1 |
| NY-SAR-35 | NY-SAR-35 | NM_152578 | CT 37 |
| OIP5 | OIP5 | NM_007280 | CT 86 |
| OTOA | Otoancorin | NM_144672 | CT 108 |
| PAGE | PAGE5 | NM_130467 | CT 16.1 |
| PAGE | PAGE5 | NM_001013435 | CT 16.1 |
| PAGE | PAGE1 | NM_003785 | CT 16.3 |
| PAGE | PAGE2 | NM_207339 | CT 16.4 |
| PAGE | PAGE2B | NM_001015038 | CT 16.5 |
| PAGE | PAGE3 | NM_001017931 | CT 16.6 |
| PAGE | PAGE4 | NM_007003 | CT 16.7 |
| PBK | PBK | NM_018492 | CT 84 |
| PIWIL2 | PIWIL2 | NM_018068 | CT 80 |
| PLAC1 | PLAC1 | NM_021796 | CT 92 |
| POTE | | NM_207355 | CT 104.5 |
| POTE | POTED | NM_174981 | CT 104.1 |
| POTE | POTEE | NM_001083538 | CT 104.2 |
| POTE | POTEA | NM_001002920 | CT 104.3 |
| POTE | POTEG | NM_001005356 | CT 104.4 |
| POTE | POTEC | NM_001137671 | CT 104.6 |
| POTE | POTEB | NM_001136213 | CT 104.7 |
| PRM1 | PRM1 | NM_002761 | CT 94.1 |
| PRM2 | PRM2 | NM_002762 | CT 94.2 |
| RBM46 | RBM46 | NM_144979 | CT 68 |
| RHOX | RHOXF2 | NM_032498 | CT 107 |
| ROPN1 | ROPN1 | NM_017578 | CT 91 |
| ROPN1 | ROPN1 | NM_031916 | CT 91 |
| SAGE | SAGE1 | NM_018666 | CT 14 |
| SEMG | SEMG1 | NM_003007 | CT 103 |
| SEMG | SEMG2 | NM_003008 | CT 103 |
| SGY-1 | SGY-1 | NM_014419 | CT 34 |
| SLCO6A1 | SLCO6A1 | NM_173488 | CT 48 |
| SPA17 | SPA17 | NM_017425 | CT 22 |
| SPACA3 | SPACA3 | NM_173847 | CT 54 |
| SPAG9 | SPAG9 | NM_003971 | CT 89 |
| SPANX | SPANXB1 | NM_032461 | CT 11.2 |
| SPANX | SPANXC | NM_022661 | CT 11.3 |
| SPANX | SPANXD | NM_032417 | CT 11.4 |
| SPANX | SPANXN5 | NM_001009616 | CT 11.10 |
| SPANX | SPANXN1 | NM_001009614 | CT 11.6 |
| SPANX | SPANXN2 | NM_001009615 | CT 11.7 |
| SPANX | SPANXN3 | NM_001009609 | CT 11.8 |
| SPANX | SPANXN4 | NM_001009613 | CT 11.9 |
| SPINLW1 | SPINLW1 | NM_181502 | CT 71 |
| SPO11 | SPO11 | NM_198265 | CT 35 |
| SPO11 | SPO11 | NM_012444 | CT 35 |
| SSX | SSX1 | NM_005635 | CT 5.1 |
| SSX | SSX2a | NM_003147 | CT 5.2a |
| SSX | SSX4 | NM_005636 | CT 5.4 |
| SSX | SSX2 | NM_175698 | CT 5.2a |
| SSX | SSX2b | NM_003147 | CT 5.2b |
| SSX | SSX3 | NM_175711 | CT 5.3 |
| SSX | SSX3 | NM_021014 | CT 5.3 |
| SSX | SSX4 | NM_175729 | CT 5.4 |
| SSX | SSX5 | NM_021015 | CT 5.4 |
| SYCE1 | SYCE1 | NM_130784 | CT 76 |
| SYCP1 | SYCP1 | NM_003176 | CT 8 |
| SYCP3 | SYCP3 | NM_153694 | CT 8 |
| TAF7L | TAF7L | NM_024885 | CT 40 |
| TAF7L | TAF7L | NM_024885 | CT 49 |
| TCC52 | TCC52 | NM_015397 | CT 102 |
| TDRD1 | TDRD1 | NM_198795 | CT 41.1 |
| TDRD3 | TDRD3 | NM_030794 | CT 41.3 |
| TDRD6 | NY-CO-45 | NM_001010870 | CT 41.2 |
| TDRD7 | TDRD7 | NM_014290 | CT 41.7 |
| TDRKH | TDRD2 | NM_006862 | CT 41.2 |
| TEX15 | TEX15 | NM_031271 | CT 42 |
| TFDP3 | HCA661 | NM_016521 | CT 30 |
| THEG | THEG | NM_016585 | CT 56 |
| TPTE | TPTE | NM_013315 | CT 44 |
| TPTE | TPTE | NM_199261 | CT 44 |
| TSGA10 | TSGA10 | NM_025244 | CT 79 |
| TSGA10 | TSGA10 | NM_182911 | CT 79 |
| TSGA10IP | TSGA10IP | NM_152762 | CT 79.10 |
| TSGA13 | TSGA13 | NM_052933 | CT 79.13 |
| TSP50 | | NM_013270 | CT 20 |
| TSPY1 | TSPY1 | NM_003308 | CT 78 |
| TSSK2 | TSSK2 | NM_053006 | CT 72.2 |
| TSSK6 | TSSK6 | NM_032037 | CT 72 |
| TSSK6 | TSSK6 | NM_032037 | CT 72 |
| TTK | TTK | NM_003318 | CT 96 |
| TULP2 | TULP2 | NM_003323 | CT 65 |
| XAGE | XAGE-2 | NM_130777 | CT 12.2 |
| XAGE | XAGE-3a v1 | NM_130776 | CT 12.3a |
| XAGE | XAGE-3a v2 | NM_130776 | CT 12.3a |
| XAGE | XAGE-1a | NM_020411 | CT 12.1a |
| XAGE | XAGE-1b | NM_020411 | CT 12.1b |
| XAGE | XAGE-1c | NM_020411 | CT 12.1c |
| XAGE | XAGE-1d | NM_020411 | CT 12.1d |
| XAGE | XAGE2 | NM_130777 | CT 12.2 |
| XAGE | XAGE3 | NM_133179 | CT 12.3a |
| XAGE | XAGE-3b | NM_130776 | CT 12.3b |
| XAGE | XAGE5 | NM_130775 | CT 12.5 |
| ZNF165 | ZNF165 | NM_003447 | CT 53 |
| | LAGE-1b | NM_020994 | CT 6.2b |
| | PASD1 | NM_173493 | CT 63 |

As those in the art will appreciate, immunoassay formats are particularly preferred for implementing the instant invention. Immunoassays can provide qualitative, semi-quantitative, or quantitative output. Immunoassays are biochemical tests that measure the presence and/or level of one or more substances, i.e., analytes, in a biological sample (which may be more readily analyzed following a fractionation or purification procedure, for example, separation of whole blood into serum or plasma components), typically a bodily fluid such as blood, serum, or urine, using the reaction of an antibody or antibodies to its antigen. The assay takes advantage of the specific binding of an antibody to its antigen. Antigens or antibodies can be detected or measured; in the context of the invention it is generally serum antibodies reactive with disease-associated antigens, e.g., anti-CT antigen autoantibodies, that are detected.

Numerous immunoassay formats are known to those of skill in the art, who understand that the signals obtained from an immunoassay are a direct result of complexes formed between one or more antibodies and polypeptides containing the necessary epitope(s) to which the antibodies bind. As used herein, the term “relating a signal to the presence or amount” of an analyte reflects this understanding. As already described, assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Because an antibody epitope is on the order of 8 amino acids, an immunoassay configured to detect two or more disease-associated autoantibody species will also detect molecules related to the native antigen, so long as those molecules (e.g., peptides, polypeptides, protein fragments, etc.) contain the epitope(s) necessary to bind to the serum antibody or antibodies being assayed. The term “related antigen” as used herein with regard to a proteinaceous biomarker refers to one or more fragments, variants, etc., of a particular marker or its biosynthetic parent that may be detected as a surrogate for the antigen itself.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation, including but not limited to, Beckman ACCESS®, Abbott AXSYM®, Roche ELECSYS®, Dade Behring STRATUS® systems, are among the immunoassay analyzers that are capable of performing immunoassays. But any suitable immunoassay may be utilized.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. Antibodies or other capture reagents (e.g., autoantibody detection reagent species) may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Detectable labels may include molecules that are themselves detectable (e.g., fluorescent moieties, electrochemical labels, ecl (electrochemical luminescence) labels, metal chelates, colloidal metal particles, etc.), as well as molecules that may be indirectly detected by production of a detectable reaction product (e.g., enzymes such as horseradish peroxidase, alkaline phosphatase, etc.) or through the use of a specific binding molecule which itself may be detectable (e.g., a labeled antibody that binds to the second antibody, biotin, digoxigenin, maltose, oligohistidine, 2,4-dintrobenzene, phenylarsenate, ssDNA, dsDNA, etc.).

Generation of a signal from the signal development element can be performed using various optical, acoustical, and electrochemical methods well known in the art. Examples of detection modes include fluorescence, radiochemical detection, reflectance, absorbance, amperometry, conductance, impedance, interferometry, ellipsometry, etc. In certain of these methods, the solid phase antibody is coupled to a transducer (e.g., a diffraction grating, electrochemical sensor, etc) for generation of a signal, while in others, a signal is generated by a transducer that is spatially separate from the solid phase antibody (e.g., a fluorometer that employs an excitation light source and an optical detector). This list is not meant to be limiting. Antibody-based biosensors may also be employed to determine the presence or amount of analytes that optionally eliminate the need for a labeled molecule.

To obtain quantitative or semi-quantitative results, results must be compared to standards of a known concentration. This is usually done though the use of one or more standard curves. The position of the curve at response of the unknown is then examined, and so the quantity of the unknown found.

Detecting the quantity of antibody or antigen can be achieved by a variety of methods, any of which can be readily adapted for practice of the invention. ELISA is a commonly used technique for detecting antibody or antigen levels. One of the most common methods is to label either the antigen or antibody with an enzyme, radioisotope, or fluorescence. Other suitable techniques include agglutination, flow cytometry, Luminex assay, cytometric bead array, and lateral flow, among others now know or later developed.

Immunoassays can involve “sandwich” approaches in which the analyte to be detected (e.g., a serum antibody reactive with a disease-associated antigen) is bound by two other entities, for example, by a capture reagent (e.g., a CT antigen reagent) immobilized on a substrate and specific for the target autoantibody species and a labeled detection reagent that binds to serum antibodies from the species to which the subject belongs (e.g., a labeled mouse antibody that reacts with human IgG). In this way the “sandwich” can be used to measure the amount of autoantibody bound between the capture and detection reagents. Sandwich assays are especially valuable to detect analytes present at low concentrations or in complex solutions (e.g., blood, serum, etc.) containing high concentrations of other molecules. As is known, in these sorts of assays a “capture” reagent (here, a disease-associated antigen such as a CT antigen or a derivative thereof that maintains or possesses an epitope recognized by the target autoantibody) is immobilized on a solid phase (i.e., on a substrate) such as a glass slide, plastic strip, or microparticle. A liquefied biological sample (e.g., serum) known or suspected to contain the target serum antibody is then added and allowed to complex with the immobilized capture reagent. Unbound products are removed and the detection reagent is then added and allowed to bind to autoantibody species that have been “captured” on the substrate by the capture reagent, thus completing the “sandwich”. These interactions are then used to quantitate the amount of autoantibody present in the biological sample.

As will be appreciated, a plurality of different disease-associated capture reagent species (including 1, 2, 5, 10, 25, 50, 100, or more CT antigen reagent species) can be immobilized on the substrate (or on different substrates, for example, different distinguishable microparticles) in order to detect, via “capture”, a plurality of different autoantibody species in a single multiplex assay. However, because each of the autoantibodies, while specific for only one particular disease associated antigen, necessarily comes from the same biological sample, and thus the same species, all of the serum antibody species bound to the substrate(s) can be detected using a common detection reagent, for example, a labeled murine monoclonal antibody specific for human IgG molecules. To allow simultaneous detection of multiple autoantibodies in a single assay, a multiplex assay format can be used. Multiplex formats provide an array of different moieties that allow simultaneous detection of multiple analytes (e.g., serum antibodies) at multiple array addresses on a single substrate. Alternatively, when a panel of the invention is spread across multiple substrates, for example, in embodiments where different disease-associated autoantibody detection reagent species are immobilized on substrates that can be distinguished (e.g., differentially labeled microparticles configured for use in Luminex assays), multiple array addresses can still be readily distinguished.

Thus, in certain embodiments, the assay methods of the invention utilize immunoassays. In certain embodiments, reagents for performing such assays are provided in an assay device, and such assay devices may be included in such a kit. Preferred reagents can comprise two or more independently selected solid phase autoantibody detection reagents, each of which comprises an antigen reagent species specific for its target autoantibody, immobilized on the same or different substrate (here, any suitable solid support). In the case of sandwich immunoassays, such reagents can also include one or more detectably labeled antibodies, the detectably labeled antibody comprising antibody that detects the intended biomarker target(s) bound to a detectable label. Additional optional elements that may be provided as part of an assay device are described hereinafter. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994.

Preparation of Substrates, Solid Phases, and Detectable Label Conjugates Often Comprise the Use of Chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In preferred embodiments a panel of the invention will also include controls, preferably at least one positive and one negative at least one positive control. Any suitable set of controls can be selected. With regard to a positive control, or set of positive controls, a particularly preferred class of positive controls are those that detect serum antibodies expected to be found in most, if not all, subjects, including those having a disease as well as those who are healthy. Examples include autoantibodies directed against antigenic components of vaccine compositions routinely administered to patients over time. Such vaccines include those against tetanus, polio, mumps, rubella, diphtheria, and measles. As a representative example, a panel of the invention can include tetanus toxoid as a detection reagent to measure reactive autoantibodies as a positive control.

Certain aspects of the present invention concern kits. Such kits comprise autoantibody detection panels according the invention in order to allow performance of the methods of the invention. As such, such kits can also include devices and instructions for performing one or more of the methods described herein. The instructions can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, computer storage media, as well as writing imprinted directly on kits.

Additional clinical indicia may be combined with the autoantibody assay result(s) of the present invention. These include other biomarkers associated or correlated with cancer. Other clinical indicia which may also be combined with the assay result(s) of the present invention includes patient demographic information (e.g., weight, sex, age, race, smoking status), medical history (e.g., family history, type of surgery, pre-existing or previous diseases), and genetic information. Combining assay results/clinical indicia in this manner can comprise the use of multivariate logistical regression, loglinear modeling, neural network analysis, n-of-m analysis, decision tree analysis, etc. This list is not meant to be limiting.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, of the present invention, optionally together with other clinical characteristics, to arrive at a diagnosis (that is, the occurrence or nonoccurrence) of cancer for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient.

In preferred diagnostic embodiments, the methods of the invention allow for diagnosing the occurrence or nonoccurrence of a disease, particularly cancer, and the assay result(s) is/are correlated to the occurrence or nonoccurrence of the particular disease. For example, each of the measured autoantibody concentration(s) may be compared to a threshold value, which may be different for each autoantibody species (or other analyte or biomarker to be studied in a given assay). The terms “correlating”, “correlated with”, and “associated with” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy that is highly efficacious and has a low level of risk, few tests are needed because clinicians and patients are willing to accept substantial diagnostic uncertainty. On the other hand, in situations where treatment options are less effective and more risky, clinicians and patients often require a higher degree of diagnostic certainty before adopting a particular treatment regimen. Thus, cost/benefit analysis is involved in selecting a diagnostic threshold.

A variety of methods may be used by to arrive at a desired threshold value for use in these methods. For example, the threshold value may be determined from a population of normal subjects by selecting a concentration representing the 75^{th}, 85^{th}, 90^{th}, 95^{th}, or 99^{th} percentile of the biomarker measured in such normal subjects. Alternatively, the threshold value may be determined from a “diseased” population of subjects, e.g., those suffering from a disease such as a cancer or having a predisposition for cancer, its recurrence, or progression, by selecting a concentration representing the 75^{th}, 85^{th}, 90^{th}, 95^{th}, or 99^{th} percentile of the biomarker measured in such subjects. In another alternative, the threshold value may be determined from a prior measurement of the biomarker in the same subject, where a prior “baseline” result is used to monitor for temporal changes in a biomarker level; that is, a temporal change in the level of the biomarker in the subject may be used for diagnostic and/or prognostic purposes.

The foregoing discussion is not meant to imply, however, that the levels of biomarkers measured in assays of the invention must be compared to corresponding individual thresholds. Methods for combining assay results can comprise the use of multivariate logistical regression, loglinear modeling, neural network analysis, n-of-m analysis, decision tree analysis, calculating ratios of markers, etc. This list is not meant to be limiting. In these methods, a composite result that is determined by combining individual biomarker data or results may be treated as if it is itself a marker; that is, a threshold may be determined for the composite result as described herein for individual biomarkers, and the composite result for an individual patient compared to this threshold.

Population studies may also be used to select a decision threshold. Receiver Operating Characteristic (“ROC”) arose from the field of signal detection theory developed during World War II for the analysis of radar images, and ROC analysis is often used to select a threshold able to best distinguish a “diseased” subpopulation from a “nondiseased” subpopulation. A false positive in this case occurs when a subject tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity versus (1—specificity) plot. A perfect test will have an area under the ROC curve of 1.0; a random test will have an area of 0.5. A threshold is selected to provide an acceptable level of specificity and sensitivity.

Thus, the ability of a particular test to distinguish two populations can be established using ROC analysis. For example, ROC curves established from a “first” subpopulation which is predisposed to future disease or disease-related changes, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured concentration of one or more target biomarkers (e.g., disease-associated serum autoantibodies), or a composite of results, may be treated as continuous variables. For example, any particular concentration can be converted into a corresponding probability of some outcome for the subject. In yet another alternative, a threshold that can provide an acceptable level of specificity and sensitivity in separating a population of subjects into “bins” such as a “first” subpopulation (e.g., which is predisposed to one or more future changes in disease status, the occurrence or recurrence of disease, a risk classification, etc.) and a “second” subpopulation which is not so predisposed. A threshold value is selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;

a specificity of greater than 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

a sensitivity of greater than 0.5, preferably at least about 0.6, more preferably at least about 0.7, still more preferably at least about 0.8, even more preferably at least about 0.9 and most preferably at least about 0.95, with a corresponding specificity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

at least about 75% sensitivity, combined with at least about 75% specificity;

a positive likelihood ratio (calculated as sensitivity/(1-specificity)) of greater than 1, at least about 2, more preferably at least about 3, still more preferably at least about 5, and most preferably at least about 10; or

a negative likelihood ratio (calculated as (1-sensitivity)/specificity) of less than 1, less than or equal to about 0.5, more preferably less than or equal to about 0.3, and most preferably less than or equal to about 0.1.

Multiple thresholds may also be used to assess patient status. For example, a “first” subpopulation that is predisposed to cancer, the recurrence of cancer, metastasis, etc., and a “second” subpopulation that is not so predisposed can be combined into a single group. This group is then subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer a negative outcome in comparison to someone in the first tertile. The third tertile is also assigned an odds ratio that is relative to that first tertile.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

### Applications

The autoantibody detection panels, arrays, and kits of the invention have numerous applications, including to monitor, prognose, diagnose, or in conjunction with treatment of a subject or patient having a disease for which the particular array is configured.

The arrays of the invention can be used to assess biological samples from patients known to have, suspected of having, or to have been previously diagnosed and/or treated for having, a particular disease, for example, a cancer such as breast cancer, as well as to screen subjects not previously known or suspected to have a particular disease. At the time of screening, the subject or patient may be symptomatic or asymptomatic. Autoantibody levels corresponding to some or all of the autoantibody-reactive reagent species, or antigens, disposed on the array can be used prognostically, for example, to determine if a patient's disease is amenable to a particular treatment, to monitor disease progression and/or effectiveness of a therapeutic regimen, to assess disease aggressiveness of disease, and/or to identify likelihood of recurrence. The arrays of the invention can also be employed for diagnostic and screening purposes. For example, arrays can be configured to use in diagnosing one or more cancers, including leukemias, melanomas, myelomas, sarcomas, and/or breast, lung, prostate, pancreatic, bladder, head and neck, colon, colorectal, and ovarian cancer.

The devices and arrays of the invention can also be used as a companion diagnostic, for example, to identify patients as likely responders or non-responders to a particular drug treatment or other therapeutic regimen, as well as for assessing the stage of a patient's disease as autoantibody profiles are likely to change during disease progression. For example, tumors express different proteins (and thus produce different antigens) to meet the different requirements at each phase of development. Similarly, autoimmune diseases can “flare” at different times.

Data sets from diseased samples can also be correlated with clinical data. Antibody profiles can be used to predict disease severity or clinical outcome, which will be useful for prognostic applications. The use of autoantibody panels will allow different stages of disease to be assessed, as the autoantibody profile of a given sample will allow the particular stage of a given disease to be discerned, thereby allowing the most effective therapeutic intervention(s) to be employed.

The devices and arrays of the invention will also find use in drug development, both in the discovery and clinical development phases, particularly for biologic drugs such as antibodies and other recombinant proteins as well as cell- or vesicle-based drug delivery systems. Drugs of this class can, at least in some cases, elicit immune responses that can be advantageous (e.g., positive response to a cancer vaccine) or harmful (e.g., severe adverse autoimmune reaction). Similarly, immune responses can also result from the administration of small molecule drugs, as a result of changes to cells and tissues following administration of the drug. The ability to monitor immune responses to biologic and small molecule drugs in clinical trials has never been more important. There is value in monitoring not only cellular immune responses but also humoral immune responses, and comparison of serum antibody profiles before and after treatment can help predict a favorable drug response. Positive responders to a drug will exhibit a different baseline humoral immune status to their disease. This is especially valuable in the case of immunomodulator class drugs that work by modifying an existing immune response rather than stimulating one de novo. By comparing data sets from non-responders to those who respond positively or negatively to a particular drug (or drug combination), panels can be defined for analyzing different groups of autoantibodies. Such panels will allow the identification of patients likely to respond to a particular therapy. Similarly, differences between responders and non-responders in the response profiles for a particular autoantibody can be used to assess whether a patient is benefiting from a particular therapeutic regimen.

As will be appreciated, different clinical study designs will allow the development of autoantibody biomarker panels that address different needs within drug development and therapy. For example, identifying responders versus non-responders will allow clinicians to select responders prior to treatment through the use of a companion diagnostic test based on response-predictive autoantibody panel profile. Similarly, to select patient cohorts in clinical trials, autoantibody profiles predictive for a positive drug response can be used to screen subjects prior to their recruitment into a clinical trial. This will ensure that only suitable candidates are included, and it may also be useful in gaining early drug approval. Also, information on drug non-response can assist regulatory bodies during consideration of drugs for approval or during post-approval surveillance (i.e., during a Phase IV clinical trial).

Another area of drug development where the instant invention will find application is in the area of “drug rescue” by helping to define the patient population(s) amenable to successful treatment as well as those who are unlikely to respond, or perhaps even more important, those who will experience an adverse reaction if administered the drug. In other words, a retrospective analysis of patient samples from a drug candidate that failed at some point in clinical development can be used to define the autoantibody panel profile(s) (or signature(s)) predictive of a positive drug response. That information can then be used to define subsequent patient cohorts for further study and treatment. This process, which may be iterated, can revitalize drugs that have fallen out of conventional clinical development due to poor or insufficient evidence of efficacy. The autoantibody panel profile(s) predictive of a positive drug response can then be used to reselect likely responders, which can lead to further clinical evaluation of the previously failed drug candidate but with a much greater likelihood of ultimately achieving drug approval.

### EXAMPLES

The following Examples are provided to illustrate certain aspects of the present invention and to aid those of skill in the art in its practice. These Examples are in no way to be considered to limit the scope of the invention in any manner, and those having ordinary or greater skill in the applicable arts will readily appreciate that the specification thoroughly describes the invention and can be readily applied to carry out the objects and obtain the ends and advantages mentioned, as well as those inherent therein.

### Example 1

### Automated Assays Using Autoantibody Detection Arrays

### A. Introduction

The following example addresses the problems of how to provide high content, high throughput, reliable identification of serum autoantibodies to tumor antigens through the provision of CT-antigen protein microarrays and an associated methodologies for effective assaying of human serum. Clones of full-length genes for many known CT antigens (see Table 2, above) have been obtained and expressed in insect cells. Each protein antigen is thus the product of a human gene, is full length, and has eukaryotic glycosylation by virtue of insect cell expression. Each of these factors is important in maintaining an authentic set of epitopes such recognition by serum autoantibodies is optimized. In order to assign statistical significance to candidate biomarker panels it is essential to assay as many serum samples as possible. Conventional techniques such as ELISA are limited in their throughput leading to what has been described as a “biomarker bottleneck”. This problem has been overcome by devising a system of automated liquid handling that ensures rapidity and tight control of the profiling assay. For this, assays were developed using a commercially available automated hybridization station (Tecan). High reproducibility was achieved by optimizing assay conditions to ensure appropriate incubation times, temperatures, sample volumes and dilutions.

The serum profiling assay devices and systems described in this example provides for the multiplex detection of serum autoantibodies to key tumor antigens. These assays utilize protein microarrays that contain protein antigens (examples of suitable autoantibody-reactive reagent species) immobilized at known discrete, independent locations on a solid substrate, which are known to be immunogenic and associated with cancer. The use of automated liquid handling equipment provides high reproducibility, high throughput, and small sample volumes (on the microliter scale or smaller). The assays of the invention have sufficient sensitivity and dynamic range to measure physiological levels of multiple serum autoantibodies in a single assay, and therefore can be used as effective tools in the measurement of humoral immune responses in diseases such as cancer as well as autoimmune disease and diseases caused by infectious agents (e.g., pathogenic bacteria, fungi, viruses, prions, and the like).

### B. Protein Array

A preferred protein microarray for the detection of tumor-associated antigens contains approximately 100 tumor antigens. Each antigen is derived from sequence-verified full-length human clones. These are expressed in insect cells and the consequent eukaryotic glycosylation helps to ensure authentic epitopes are maintained. The proteins were selected from known tumor-associated antigens. The microarrays of the invention can readily be adapted to include additional or alternative tumor-associated or other antigens now known or later discovered. In designing the microarrays used in the assays described in these examples, the criteria for inclusion on the microarray included demonstrable immunogenicity, association with cancer, and suitability for use as biomarkers. In addition to tumor antigens, a protein microarray preferably also contains one or more control features, as described below.

### 1. Control Features on a Preferred Tumor Antigen Protein Array

a. Parent vector lysate: This negative control ensures that signals from the assay are not the result of non-specific binding (i.e., serum autoantibody cross-reactivity) to insect cell proteins that may be carried through the purification procedures used to purify tumor antigens expressed in a suitable eukaryotic expression system, for example, a baculovirus expression system.
b. Sheep IgG: This negative control is spotted on the array substrate at 500 ng/μL to ensure specificity of anti-human-IgG detector antibody. Signals from this feature should be negative when probed with a detector antibody species.
c. This positive control, which is preferably spotted on the array substrate at various concentrations, for example, 500 ng/μL, 100 ng/μL, 10 ng/μL, 1 ng/μL, and 0.1 ng/μL, ensures specificity of anti-human-IgG detector antibody. Signals from this feature should be positive when probed with detector antibody. Moreover, positive signals from this feature can be used to quantitatively assess serum autoantibody concentrations for tumor (or other) antigens spotted or immobilized on the substrate, as signals from this positive control feature can be used to develop a standard curve correlated with antigen: antibody concentration.
d. Cy5-BSA: This positive control, which is spotted at 1/1000 dilution, ensures scanning conditions are correct. The fluorescent signal from this feature should be consistent and provides “landing light” for orientation.

### 2. Quality Control

To ensure quality control, the processes for producing protein microarrays and performing assays using such microarrays have been analyzed to identify the critical control points (CCPs). Each CCP can be made the subject of appropriate QC control measures (see below).

a. DNA QC: Only full-length sequence-verified human clones of desired tumor antigens (or other disease-associated antigens) are used to produce proteins for immobilization on a protein microarray. Preferably, at least the antigen-coding regions of expression vectors are confirmed by sequencing prior to use for expression of the desired autoantibody antigen.
b. Protein QC: Following expression of a desired autoantibody antigen, the protein expression products are characterized to ensure production of the desired antigen. Characterization can be by SDS-PAGE gel electrophoresis and Western Blotting, peptide mapping, or the like to ensure that sufficient protein having the predicted mass has been produced.
c. Array QC: From each manufactured batch, protein microrarrays are taken from the beginning, middle, and end of the production run for testing. In preferred embodiments, testing includes probing a protein microarray with antibody to, for example, cMyc to verify excess protein deposition in each desired location (or spot) on the surface of the substrate, as each target antigen can be engineered to express the cMyc (or other desired) epitope. In such embodiments, the fusion protein that comprises the QC epitope (e.g., a cMyc epitope) also comprises the target antigen. In one alternative approach, the expression products that comprise a target antigen can include a “tag”, peptide linker, or like structure at the N- or C-terminus of target antigen in order to provide a moiety to which a QC epitope or element can be linked (covalently o non-covalently).

### C. Protocol Description

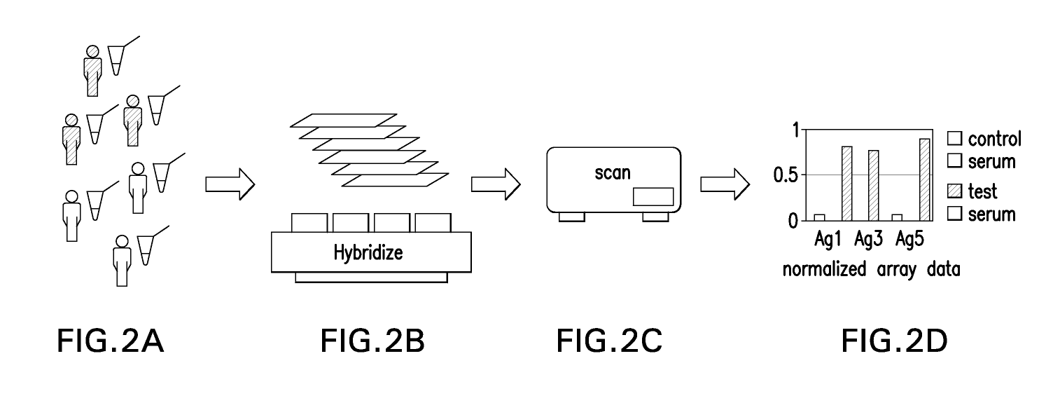

An autoantibody detection array can be disposed in an automated liquid-handling and hybridization station (Tecan) to ensure assay consistency. Alternatively, autoantibody detection arrays can be embodied in single-use lateral flow devices configured to allow autoantibodies in serum to bind to their corresponding tumor antigen (each a different autoantibody-reactive reagent species) on the protein microarray disposed in a detection zone in the device. Serum autoantibody species complexed with their target antigens can be visualized using any suitable detection system configured to detect bound serum autoantibody species. Particularly preferred are systems configured to detect a fluorescent detector antibody specific for human serum antibodies immobilized to their respective target antigens spotted on the protein microarray. The location and strength of signals detected by the detection system allows a determination of which autoantibody species is/are present in the particular sample being analyzed. If desired, the system can be configured for qualitative, semi-quantitative, or quantitative detection of autoantibodies. FIG. 2 illustrates an overview of an assay according to the invention. In part (A), samples are obtained from each of several patients. An aliquot from a patient sample (which may or may not be diluted, for example, a 1:10 dilution) is placed in a test device (such as a suitably configured Tecan system or lateral flow device) that allows the sample to incubate and thus interact with the autoantibody-reactive reagent species disposed on the array substrate in the detection zone of the device. FIG. 2(B). Autoantibodies bound to target antigens are then detected using a suitably configured detector (the “scanner” shown in FIG. 2(C)) such as a microarray fluorescence scanner (Perkin Elmer). Detector antibodies may be included in a device according to the invention or they may be added after the sample. One or more washing steps may be employed although washing is not necessary, between the incubation and detection steps. The results are then analyzed and output in a desired fashion, for example, by graphic representation (FIG. 2(D)) showing normalized amounts of autoantibody detected at the locations for particular autoantibody-reactive reagent species (e.g., “Ag1”, “Ag3”, “Ag5”) disposed on the array substrate.

### D. Reproducibility

For effective biomarker discovery it is preferred that autoantibody detection arrays of the invention and assays that employ them achieve a high standard of reproducibility so that subtle changes in serum profiles can be reliably detected. A number of measures have been taken to ensure high assay reproducibility. For example, the use of automated liquid handling systems ensures that all volumes of sample and buffer are precise and consistent throughout a particular assay and from assay-to-assay. In some preferred embodiments, target protein antigens are immobilized onto the array in duplicate or triplicate so that spot-to-spot (intra-array) variability can be measured. In addition, the variability between different autoantibody detection arrays disposed on the same substrate (inter-array) and between slides (inter-slide, inter-assay) can be measured and CV values recorded. An example of such intra-assay and inter-array, inter-slide assessments is shown in Table 3, below.

TABLE 3
Reproducibility of profiling assay - CV values

| | | min | max | average |
|---|---|---|---|---|
| spot-to-spot | intra-array | 3.1 | 27.2 | 15.15 |
| array-to-array | inter-array | 6.4 | 35.8 | 21.1 |
| (within slide) | | | | |
| slide-to-slide | inter-slide | 7.6 | 37.3 | 22.45 |

### E. Sample Handling

Serum samples are preferably stored at −20° C. prior to assay. If necessary, samples can be shipped on dry ice in 200 μL aliquots using a suitable container (e.g., 0.5 mL or 1.5 mL cryotube or Eppendorf microcentrifuge tube). Freeze thaw cycles should be kept to a minimum.

### F. Sensitivity and Dynamic Range

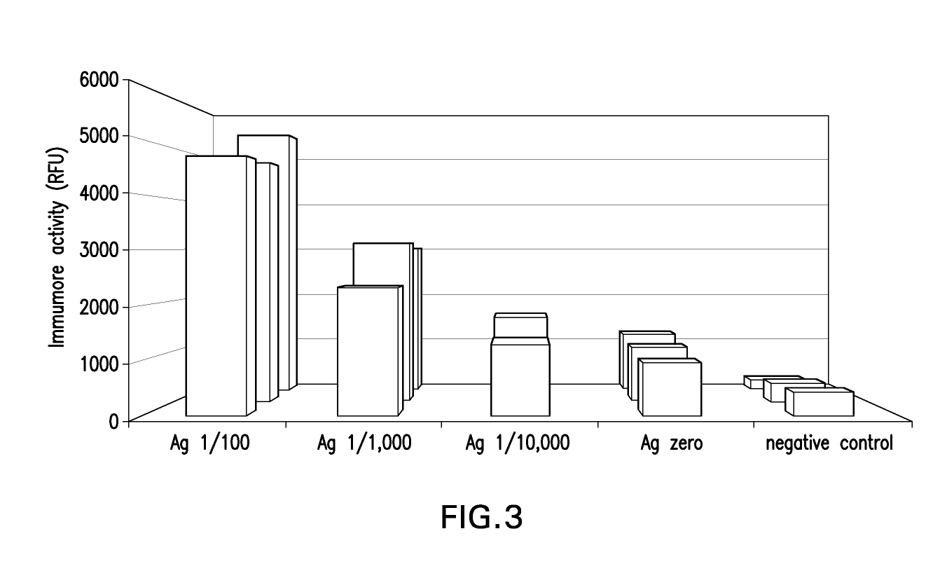

The sensitivity of an autoantibody detection array according to the invention should be sufficient for the detection of physiological quantities of autoantibody species yet have a sufficient dynamic range not to be saturated when serum autoantibodies are elevated. These parameters can be tested, for example, by assaying a series of samples that have been spiked with defined quantities of antibody. The results of such a test are shown in FIG. 3.

The test for which results are shown in FIG. 3 involved an experiment using a protein microarray that comprised the tumor-associated protein p53 immobilized as the autoantibody-reactive reagent species on the surface of the array substrate. The microarray was probed with human serum samples that had been spiked with different serially diluted concentrations of antibody specifically reactive with p53. The antibody dilutions were performed by serial 10-fold dilutions from a working stock of 2 mg/mL anti-p53 antibody to attain final concentrations of 20.0 μg/mL (1:100 dilution), 2.0 μg/mL (1:1,000 dilution), and 0.2 μg/mL (1:10,000 dilution). The background signal in the assay was typically 200-500 RFU (Relative Fluorescent Units). The presence of antibody was recorded as positive if the signal from the corresponding antigen on the array was at least three times greater than the background signal. In this test, using these parameters, the minimal level of antibody that was detectable was ˜5 μg/mL. It has been reported that humoral immune responses result in the production of specific antibodies in the range of 10-100 μg/mL. In addition, the gradient of response indicates that there is sufficient antigen on the array for the signal not to be saturated, even with excessive (greater than physiological) quantities of serum antibody.

In conclusion, the microarrays of the invention and assays that employ them have sufficient sensitivity and dynamic range for the profiling of humoral immune responses to tumor antigens.

### Example 2

### Autoantibody Detection In Melanoma Patients

Autoantibody detection arrays as described in Example 1 were used to assay serum samples from 50 patients with advanced Stage IV metastatic melanoma for antibodies to CT antigens. Patients with this disease were found to have autoantibodies to fifty CT antigens in contrast to normal healthy serum controls. Autoantibodies that were detected in the patient samples included those reactive with the following CT antigens: CTAG2, MAGEA4v2, MAGEA5, MAGEA11, NLRP4, LIP1, MAGEB6, BAGE5, MAGEB5, BAGE2, DSCR8/MMA1, DDX53, NY-ESO-1, PBK, MICA, CXorf48.1, CT47.11, GAGE1, SSX2A, NYCO45, CSAG2, HORMAD1, ZNF165, SYCP1, GAGE5, BAGE4, SPANXD, MAGEA2, GAGE6, CEP290, NXF2, COL6A1, XAGE-2, SPANXA1, GAGE2A, SYCE1, LDHC, FTHL17, BAGE3, MAGEA4v3, MAGEB1, p53, GRWD1, MART1, MAGEA1, OIP5, CCDC33, MAGEA3, and XAGE3av2.

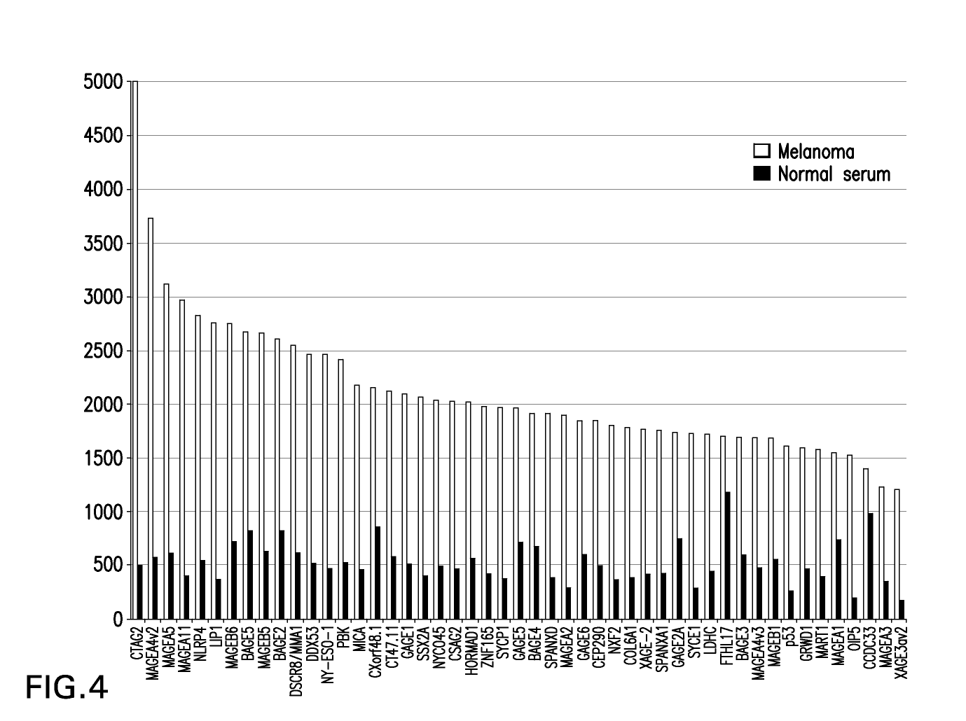

Results are also shown in FIG. 4, which plots relative autoantibody levels in serum versus autoantibody species for both melanoma patients and normal, healthy controls.

### Example 3

### Autoantibody Detection In NSCLC Patients

Autoantibody detection arrays as described in Example 1 were used to assay serum samples from three patients with advanced non-small cell lung carcinoma for antibodies to CT antigens. Patients with this disease were found to have autoantibodies to numerous CT antigens in contrast to normal healthy serum controls. Autoantibodies that were detected in the patient samples included those reactive with the following CT antigens: GAGE2A, BAGE4, BAGE2, MAGEA1, DSCR8/MMA1, CCDC33, BAGE5, CEP290, GAGE1, PBK, FTHL17, BAGE3, NLRP4, CT62, SPANXA1, DDX53, COL6A1, CSAG2, SSX2A, CT47.11, SYCP1, SPANXD, GAGE6, TSSK6, MAGEB5, ZNF165, LIP1, MICA, GAGE4, SSX4, MAGEB6, CXorf48.1, MAGEA4v2, COX6B2, MAGEA11, GRWD1, LEMD1, CTAG2, LDHC, XAGE3av2, SP011, HORMAD1, SPANXB1, TYR, MAGEB1, NYCO45, ROPN1, MAGEA5, XAGE3av1, MAGEA10, SILV, MART1, SGY-1, NXF2, MAGEA2, and RELT.

### Example 4

### Autoantibody Detection In SCLC Patients

Autoantibody detection arrays as described in Example 1 were used to assay serum samples from 29 patients with extensive stage small cell lung carcinoma (ES SCLC) for antibodies to CT antigens. Patients with this disease were found to have autoantibodies to numerous CT antigens in contrast to normal healthy serum controls. Autoantibodies that were detected in the patient samples included those reactive with the following CT antigens: BAGE4, GAGE2A, FTHL17, BAGE2, XAGE3av2, MAGEA11, GAGE6, SPANXA1, CEP290, BAGE5, CCDC33, MAGEB6, MAGEA4v2, SPANXD, LEMD1, CT47.11, BAGE3, NYCO45, MAGEA3, COX6B2, MAGEB5, GAGE5, GAGE4, MAGEA10, SYCE1, MAGEA5, MAGEA4v4, CT62, GAGE7, NLRP4, DSCR8/MMA1, NXF2, MAGEB1, ZNF165, CSAG2, DDX53, CXorf48.1, GAGE1, PBK, LDHC, HORMAD1, ROPN1, LIP1, CTAG2, SSX2A, SYCP1, MAGEA1, XAGE-2, NY-ESO-1, SPANXC, OIP5, SGY-1, SSX1, SP011, XAGE3av1, MAGEA4v3, SPANXB1, SSX4, THEG, and TSSK6.

### Example 5

### Autoantibody Detection In Colorectal Cancer Patients

Autoantibody detection arrays as described in Example 1 were used to assay serum samples from one patient with extensive stage colorectal cancer for antibodies to CT antigens. This patient was found to have autoantibodies to numerous CT antigens in contrast to normal healthy serum controls. Autoantibodies that were detected in the patient samples included those reactive with the following CT antigens: BAGE4, BAGE2, GAGE2A, FTHL17, CCDC33, BAGE5, BAGE3, CEP290, PBK, SPANXA1, GAGE1, MAGEA1, DDX53, COX6B2, XAGE3av2, CT47.11, GAGE6, CT62, GAGE5, SP011, XAGE-2, CXorf48.1, DSCR8/MMA1, GAGE4, LEMD1, MAGEA11, SYCE1, SYCP1, MAGEB6, HORMAD1, COL6A1, CSAG2, SPANXD, NYCO45, ZNF165, LDHC, GAGE7, MAGEB5, TYR, MAGEB1, SSX4, MAGEA10, CTAG2, GRWD1, THEG, XAGE3av1, MAGEA3, SPANXB1, and SPAG9.

### Example 6

### Autoantibody Detection In Sarcoma Patients

Autoantibody detection arrays as described in Example 1 were used to assay serum samples from two patients with refractory sarcoma for antibodies to CT antigens. Patients with this disease were found to have autoantibodies to numerous CT antigens in contrast to normal healthy serum controls. Autoantibodies that were detected in the patient samples included those reactive with the following CT antigens: GAGE5, BAGE4, BAGE2, GAGE2A, BAGE3, DSCR8/MMA1, CEP290, FTHL17, CXorf48.1, PBK, BAGE5, DDX53, CCDC33, MAGEA1, GAGE1, SPANXD, SYCE1, MAGEB6, SPANXA1, COL6A1, XAGE3av2, GRWD1, MAGEA4v2, MAGEA3, CT47.11, XAGE-2, CSAG2, COX6B2, SYCP1, GAGE6, MICA, CTAG2, GAGE4, NYCO45, MAGEB5, ZNF165, and TYR.

### Example 7

### Autoantibody Detection In Prostate Cancer Patients

Autoantibody detection arrays as described in Example 1 were used to assay serum samples from 62 patients with different stages of prostate cancer for antibodies to CT antigens. Patients with this disease were found to have autoantibodies to numerous CT antigens in contrast to normal healthy serum controls. Autoantibodies that were detected in the patient samples included those reactive with the following CT antigens: NLRP4, BAGE4, BAGE5, HORMAD1, CT47.11, MAGEB6, GAGE6, MAGEA11, CTAG2, CCDC33, NYCO45, MAGEB1, MAGEA4v2, GAGE1, CEP290, MAGEB5, CSAG2, MAGEA5, PBK, FTHL17, DSCR8/MMA1, LDHC, LIP1, LEMD1, TSGA10, GAGE2A, COX6B2, SPANXA1, MART1, NY-ESO-1, GAGE5, MICA, SGY-1, MAGEA2, NXF2, MAGEA10, CT62, and GRWD1.

### Example 8

### Autoantibody Detection In Ovarian Cancer

Autoantibody detection arrays as described in Example 1 were used to assay serum samples from a patient with untreatable ovarian cancer for antibodies to CT antigens. This patient had autoantibodies to numerous CT antigens in contrast to normal healthy serum controls. Autoantibodies that were detected in the patient samples included those reactive with the following CT antigens: FTHL17, CTAG2, GAGE5, GAGE6, MAGEB6, GAGE2A, SPANXA1, MAGEA5, CCDC33, MAGEA11, BAGE2, BAGE5, NLRP4, MAGEA4v2, NY-ESO-1, XAGE-2, PBK, BAGE3, COX6B2, HORMAD1, CXorf48.1, CEP290, SPANXD, NYCO45, SYCP1, CT47.11, MAGEB1, DDX53, GAGE4, MAGEB5, BAGE4, LEMD1, ZNF165, CSAG2, LIP1, GRWD1, CT62, DSCR8/MMA1, GAGE1, MAGEA10, MICA, SYCE1, SSX2A, LDHC, and XAGE3av2.

### Example 9

### Autoantibody Detection In Esophageal Cancer Patients

Autoantibody detection arrays as described in Example 1 were used to assay serum samples from patients with untreatable esophageal cancer for antibodies to CT antigens. Patients with this disease were found to have autoantibodies to numerous CT antigens in contrast to normal healthy serum controls. Autoantibodies that were detected in the patient samples included those reactive with the following CT antigens: GAGE5, CCDC33, BAGE4, BAGE5, MAGEB6, FTHL17, DSCR8/MMA1, GAGE6, SPANXA1, MAGEA5, GAGE2A, GAGE1, CEP290, CTAG2, MAGEA11, CSAG2, LEMD1, XAGE3av2, MAGEA4v2, BAGE2, NYCO45, SYCE1, MAGEB5, HORMAD1, SPANXD, NY-ESO-1, XAGE-2, LIP1, MAGEB1, GRWD1, CT47.11, MAGEA3, DDX53, MAGEA10, NLRP4, CXorf48.1, and MICA.

### Example 10

### Autoantibodies In Cancer

This example shows a compilation of data collected from analyses performed in during the experiments reported in Examples 2-9, above, along with data representing CT antigen-specific autoantibody analyses also performed on serum from three normal, healthy patients in whom cancer had never been diagnosed. The resulting autoantibody profiles are shown in Table 4, below.

TABLE 4
CT antigen autoantibody profiles in 8 cancers and normal controls

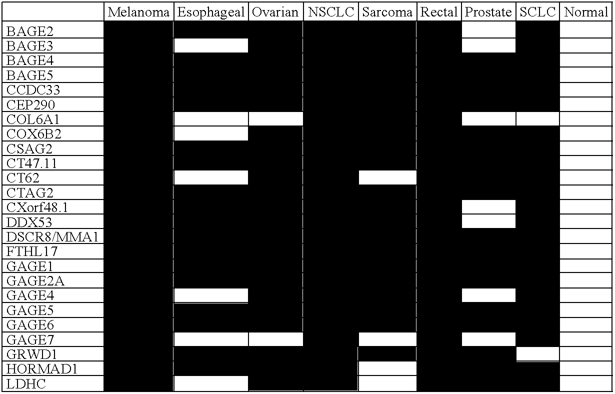

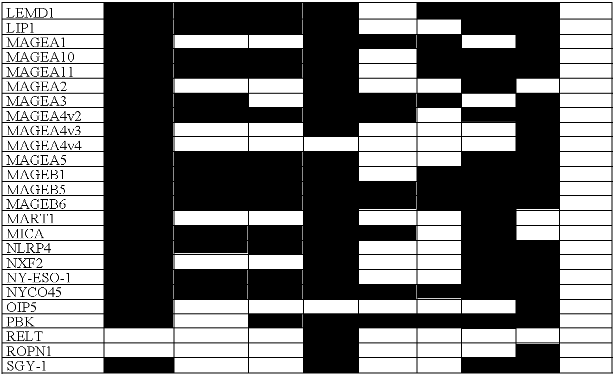

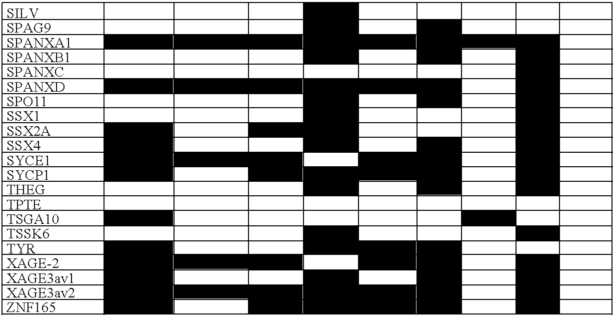

The data in Table 4 shows that different cancers exhibit different anti-CT antigen serum antibody profiles, thereby allowing not only a range of different cancers to be detected by rapid, multiplex analysis of readily obtained patient serum samples, but also to distinguish various cancer types based on serum antibody profiles to CT antigens.

All of the compositions, articles, devices, systems, and methods disclosed and claimed herein can be made and executed without undue experimentation in light of the present disclosure. While the compositions, articles, devices, systems, and methods of this invention have been described in terms of preferred embodiments, it will be apparent to those of skill in the art that variations may be applied to the compositions, articles, devices, systems, and methods without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims, which may also contain even further embodiments of the invention.

What is claimed is:

1. An autoantibody detection panel, comprising:
a-1. a plurality of independently selected autoantibody detection reagent species, wherein at least one of the autoantibody detection reagent species is a CT antigen reagent species specifically reactive with a cancer-associated autoantibody species reactive with naturally occurring cancer-associated CT antigen species; or
a-2. a plurality of independently selected autoantibody detection reagent species that are CT antigen reagent species, wherein at least first and second CT antigen reagent species are specifically reactive with first and second autoantibody species reactive with naturally occurring cancer-associated CT antigen species; and
b. at least one substrate, optionally a solid support, upon which the plurality of autoantibody detection reagent species are immobilized.

2. An autoantibody detection panel according to claim 1 that comprises from 2 to about 500 independently selected CT antigen reagent species.

3. An autoantibody detection panel according to claim 1 wherein each CT antigen reagent species comprises an autoantibody-reactive moiety selected from the group consisting of a polypeptide and a peptide, wherein the polypeptide optionally is a full-length CT antigen polypeptide, a fragment of a full-length CT antigen polypeptide that comprises a CT antigenic domain, a synthetic polypeptide that comprises a CT antigenic domain, or a peptide that comprises a CT antigenic domain.

4. An autoantibody detection panel according to claim 1 further comprising at least one of the following:
a. at least one disease-associated non-CT antigen reagent species disposed on the substrate, wherein the disease-associated non-CT antigen reagent species comprises a cancer-associated protein, optionally p53 or a p53 variant, a matrix metalloproteinase, and a mucin; and
b. at least one positive control reagent disposed on the substrate, wherein the positive control reagent is reactive with an antigen optionally selected from the group consisting of tetanus toxoid, polio virus, rubella, diphtheria, mumps, and measles.

5. An autoantibody detection panel according to claim 1 comprising a plurality of substrates, wherein each is optionally a solid support upon which a different CT antigen reagent species is immobilized.

6. An autoantibody detection panel according to claim 1 configured as an array or microarray wherein each of plurality of independently selected autoantibody detection reagent species are immobilized on (i) the same substrate or (ii) different substrates, optionally microparticles.

7. A kit comprising an autoantibody detection panel according to claim 6 and instructions for use of the array or microarray.

8. A method of analyzing a biological sample, comprising contacting under binding conditions an autoantibody detection panel according to claim 1 with an aliquot of a biological sample obtained from a subject and determining whether the biological sample contains an autoantibody reactive with at least one CT antigen reagent species of the autoantibody detection panel, thereby analyzing the sample.

9. A method according to claim 8 performed as a screen to assess whether the subject (a) is healthy or diseased, (b) has been administered a drug, or, (c) if the subject has been administered a drug, whether the subject responds favorably, detrimentally, or does not respond to the drug.

10. A method according to claim 8 performed a purpose selected from the group consisting of monitoring disease progression, disease diagnosis, disease prognosis, or disease treatment.

11. A method according to claim 10 wherein the disease is a cancer, wherein the cancer is optionally selected from the group consisting of breast cancer, a lung cancer, prostate cancer, pancreatic cancer, colon cancer, colorectal cancer, a melanoma, a myeloma, ovarian cancer, a leukemia, and a sarcoma.

12. A method according to claim 8 performed to determine an autoantibody profile of the subject, wherein the autoantibody profile is reflective of disease-associated autoantibody species present in the biological sample that are reactive with autoantibody detection reagent species of the autoantibody detection panel.

13. A method according to claim 8 wherein the determining step is performed by detecting complexes of autoantibody species present in the biological sample specifically reactive with a CT antigen reagent species of the article, wherein optionally detection of complexes is performed under binding conditions using (a) a detection reagent capable of binding to one or more autoantibody species from the subject and (b) detecting the detection reagent, wherein the detection reagent optionally comprises a detectable label and an antibody, an antibody fragment, or an antibody derivative.

14. A method according to claim 8 wherein the biological sample is selected from the group consisting of a bodily fluid or tissue sample, wherein (a) the bodily fluid is selected from the group consisting of whole blood, blood plasma, blood serum, saliva, urine, synovial fluid, cerebrospinal fluid, mucous, a nasal secretion, sputum, amniotic fluid, and bronchoalveolar lavage fluid and (b) the tissue sample comprises cells or components from of cells, in either case selected from the group consisting of peripheral blood mononuclear cells, total white blood cells, lymph node cells, spleen cells, tonsil cells, skin cells, and cells from a biopsy.

15. A method according to claim 8 further comprising assessing a change in an autoantibody parameter of at least one autoantibody capable of analysis by the method, wherein the autoantibody parameter optionally is selected from the group consisting of presence or absence of the autoantibody and a change in an amount of the autoantibody, optionally an increase or decrease in the amount of the autoantibody.

16. A method according to claim 15 to assess a change in an autoantibody parameter of a plurality of autoantibody species.

17. A method according to claim 8 wherein the biological sample is obtained from a mammalian subject, optionally a human subject.

18. A method according to claim 16 wherein the result of the analysis of autoantibody parameters is used to make an assessment selected from the group consisting of:
(a) whether the subject has, or is pre-disposed to have, a disease;
(b) whether the subject has a recurrence of a disease; and
(c) whether the subject is responsive to a disease treatment.
